(19) United States
(12) Patent Application Publication
(10) Pub. No.: US 2024/0174626 A1
(43) Pub. Date: May 30, 2024

(54) MONOMERS CONTAINING A 4-THIAZOLIDINONE UNIT AND THEIR POLYAMIDE, POLYESTER AND POLYURETHANE POLYCONDENSATES

(71) Applicant: COMPAGNIE GENERALE DES ETABLISSEMENTS MICHELIN, CLERMONT-FERRAND (FR)

(72) Inventors: Milan FEDURCO, CLERMONT-FERRAND Cedex 9 (FR); Marco RIBEZZO, CLERMONT-FERRAND Cedex 9 (FR)

(21) Appl. No.: 18/548,366

(22) PCT Filed: Feb. 21, 2022
(86) PCT No.: PCT/FR2022/050302

§ 371 Date: Aug. 30, 2023

(30) Foreign Application Priority Data
Mar. 2, 2022 (FR) 2102008

(51) Int. Cl. C07D 277/14; C08G 18/10; C08G 18/38; C08G 63/685; C08G 63/688; C08G 69/42

(52) U.S. Cl. CPC C07D 277/14; C08G 18/10; C08G 18/3857; C08G 63/685; C08G 63/688; C08G 69/42

(57) ABSTRACT

Monomers which contain a 4-thiazolidinone unit connected to a benzene nucleus and which contain two functions selected from hydroxyl and carboxyl are provided. A process for synthesizing them and to the use of them in the synthesis of polycondensates such as polyamides, polyesters and polyurethanes is also provided. Due to the monomers according to the disclosure, 2-mercaptopropionic acid is not used in the synthesis of polycondensates containing 4-thiazolidinone units.

This U.S. patent application is a national phase entry of international patent application no. PCT/FR2022/050302, filed Feb. 21, 2022, which claims priority to French patent application no. FR2102008, filed Mar. 2, 2021, the entire contents of which are incorporated herein by reference in their entirety.

### BACKGROUND

### 1. Technical Field

The field of the present invention is that of monomers comprising a 4-thiazolidinone unit, which are intended for use in the synthesis of polyamides, polyesters and polyurethanes obtained by polycondensation.

### 2. Related Art

The synthesis of polycondensates comprising 4-thiazolidinone units is described in the journal Polymer Chemistry, 2014, 5, 2695. It is carried out by polycondensation reaction of 2-mercaptopropionic acid, a diamine such as hexamethylenediamine and a dialdehyde such as terephthalaldehyde. The authors of the publication highlight that the polycondensate synthesis process is environmentally friendly, because the polycondensation reaction is carried out at ambient temperature in the absence of a catalyst and generates only water as a by-product. Nevertheless, the synthesis of the polycondensate proves to be very bothersome for the surrounding area, since it is accompanied by a very unpleasant odor due to the use of 2-mercaptopropionic acid when the latter is used in large amounts. Even though the polymers described above are thermally stable, they prove to be brittle.

### SUMMARY

The inventors have developed new monomers which make it possible to remedy these drawbacks in the synthesis of polycondensates containing 4-thiazolidinone units, by using compounds, amine or aldehyde, which are heterodifunctional. Thanks to these new monomers, no 2-mercaptopropionic acid is used in the synthesis of polycondensates containing 4-thiazolidinone units. Due to their structure, the monomers according to the invention also make it possible to obtain a wide variety of polycondensates which are polyamides, polyesters and polyurethanes and which all have the shared feature of containing 4-thiazolidinone units in their monomer units and which have improved thermal and mechanical properties compared to the polycondensate containing 4-thiazolidinone units of the prior art described above.

Thus, a first subject of the invention is a monomer which contains a 4-thiazolidinone unit connected to a benzene nucleus and which contains two functions selected from hydroxyl and carboxyl, which monomer is of formula (I-a) or (I-b) in which Y is hydroxyl or carboxyl, Q is alkyl or carboxyalkyl, R₂ is hydrogen, hydroxyl, carboxyl, alkyl or hydroxyalkyl, Y′ is hydroxyl or carboxyl; Q′ is alkyl or carboxyalkyl,

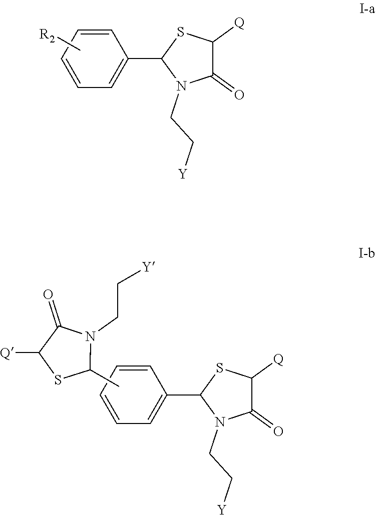

or of formula (I-c) in which Y is hydroxyl or carboxyl, Q is alkyl or carboxyalkyl, Y′ is hydroxyl or carboxyl; Q′ is alkyl or carboxyalkyl, x is an integer ranging from 0 to 2,

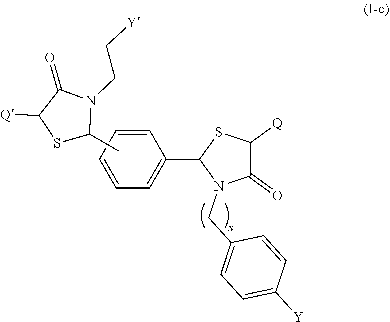

or else of formula (II-1) in which Q is alkyl or carboxyalkyl, R₃ is hydrogen, hydroxyl, carboxyl, alkyl or hydroxyalkyl, R₁ is alkyl and A₁, a divalent carbon group which contains carbon atoms, is an alkanediyl or an alkanediyl interrupted by one or more oxygen atoms, m is an integer ranging from 0 to 4.

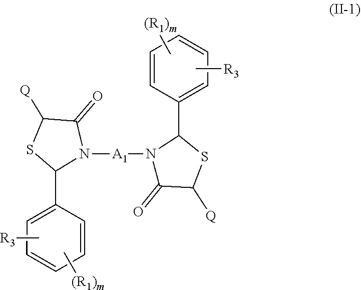

The invention also relates to a process for synthesizing a monomer in accordance with the invention, which process comprises the reaction of an aldehyde of formula (II), a compound (III) and a compound of formula (IV), the compound (III) being of formula Y-A-NH₂ or of formula H₂N-A₁-NH₂, in which Y is hydroxyl or carboxyl, Q is alkyl or carboxyalkyl, A is an ethanediyl, A₁ is an alkanediyl or an alkanediyl interrupted by one or more oxygen atoms, R₁ is alkyl, m is an integer ranging from 0 to 4, R is hydrogen, hydroxyl, carboxyl, alkyl, hydroxyalkyl or an aldehyde function.

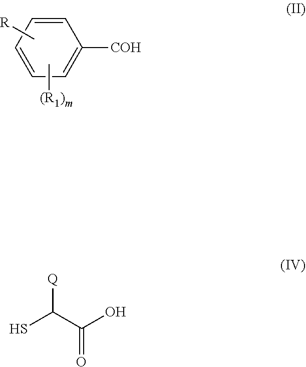

The invention also relates to processes for the synthesis of polycondensates using the monomers in accordance with the invention.

Thus a first process for synthesizing polycondensates in accordance with the invention is a process for synthesizing a polyamide which comprises the reaction of a monomer in accordance with the invention with a second monomer containing two functions selected from amine and isocyanate, the monomer in accordance with the invention containing two carboxyl functions.

A second process for synthesizing polycondensates in accordance with the invention is a process for synthesizing a polyester which comprises the reaction of a monomer in accordance with the invention with a second monomer, both monomers each containing a carboxyl function and a hydroxyl function.

A third process for synthesizing polycondensates in accordance with the invention and an alternative to the second process is a process for synthesizing a polyester which comprises the reaction of a monomer in accordance with the invention with a second monomer, one of the two monomers containing two hydroxyl functions and the other containing two carboxyl functions.

A fourth process for synthesizing polycondensates in accordance with the invention is a process for synthesizing a polyurethane which comprises the reaction of a monomer in accordance with the invention with a second monomer containing two isocyanate functions, the monomer in accordance with the invention containing two hydroxyl functions.

The invention also relates to processes for synthesizing polycondensates which differ respectively from the four processes for synthesizing polycondensates which are the first, the second, the third and the fourth process, by the partial or total replacement of the second monomer by a prepolymer which contains two reactive functions identical to that of the second monomer.

Another subject of the invention is a polycondensate capable of being obtained by any one of the processes for synthesizing polycondensates in accordance with the invention.

### DETAILED DESCRIPTION OF EXAMPLE EMBODIMENTS

The compounds mentioned in the description may be of fossil origin or may be biobased. In the latter case, they may be partially or completely derived from biomass or may be obtained from renewable starting materials derived from biomass. Similarly, the compounds mentioned may also originate from the recycling of already-used materials, i.e. they may partially or totally result from a recycling process, or else be obtained from starting materials which themselves result from a recycling process.

In the present application and in a known manner, the term hydroxyl means the OH group, the term carboxyl means the COOH function, the term carboxyalkyl means an alkyl group which is substituted by a COOH function, and the term hydroxyalkyl means an alkyl group substituted by an OH function.

In the present application, when a symbol represents a hydrogen atom, it is said in the present application that it is hydrogen.

The term carbon group means a group which contains carbon atoms, in particular a hydrocarbon group which may be substituted or interrupted by one or more heteroatoms such as oxygen, sulfur, nitrogen and silicon atoms.

When R₂ is different from a hydrogen atom, R₂ is preferentially para or meta relative to the thiazolidinone ring, more preferentially para relative to the thiazolidinone ring.

When R₃ is different from a hydrogen atom, R₃ is preferentially para or meta relative to the thiazolidinone ring, more preferentially para relative to the thiazolidinone ring.

The carbon number of the alkanediyl in A₁ can vary to a large extent. A₁ may contain 2 to 20 carbon atoms. It is preferentially a linear or branched chain. The choice of the structure of the divalent carbon group A₁ is guided by the flexibility properties desired for the polycondensate. For example, the presence of oxygen atoms within the main alkanediyl chain of A₁ for forming ether bonds is conducive to the obtaining of a relatively flexible polycondensate. A high number of carbon atoms in an alkanediyl chain also contributes to increasing the flexibility of the polycondensate. Preferentially, A₁ contains at least 4 carbon atoms.

In formula (Ia), x can take the values of 0, 1 or 2 and q is preferentially equal to 0.

The alkyl groups may be linear, branched or cyclic. They are preferentially linear. They preferentially contain 1 to 12 carbon atoms.

The alkyl groups represented respectively by the symbols Q and Q′ are preferentially methyl due to the commercial availability of one of the precursors for synthesis of the monomer in accordance with the invention, 2-mercaptopropionic acid.

The alkyl groups represented by the symbols R₁, R₂, R₃ are preferentially alkyls having 1 to 3 carbon atoms, more preferentially are methyl. It follows that the alkyl group represented by the symbol R of formula (II) of the compound useful for the synthesis of the monomer is also preferentially an alkyl having 1 to 6 carbon atoms, more preferentially is methyl.

The alkyl groups substituted by a hydroxyl group to constitute the hydroxyalkyl group represented by the symbols R₂ and R₃ are preferentially alkyl having 1 to 6 carbon atoms, more preferentially are methyl, in which case the hydroxyalkyl group is then of formula CH₂OH. It follows that the alkyl group substituted by a hydroxyl group to constitute the hydroxyalkyl group represented by the symbol R of formula (II) of the compound useful for the synthesis of the monomer is also preferentially an alkyl having 1 to 6 carbon atoms, more preferentially is methyl.

The alkyl groups substituted by a carboxyl group to constitute the carboxyalkyl groups respectively represented by the symbols Q and Q′ are preferentially alkyl having 1 to 6 carbon atoms, more preferentially are methyl, in which case the carboxyalkyl group is then of formula CH₂ COOH.

Preferentially, Q and Q′ are methyl or carboxymethyl.

Preferably, R₂ is hydrogen, hydroxyl, carboxyl or hydroxymethyl.

R₃ is preferably hydroxyl or carboxyl. When R₃ is hydroxyl or carboxyl, Q is preferentially alkyl, more preferentially methyl.

In formula (II-1), m is preferentially equal to 0.

According to a first variant of the invention, the monomer is of formula (I-a) or (I-b) in which Y is hydroxyl or carboxyl, Q is alkyl or carboxyalkyl, R₂ is hydrogen, hydroxyl, carboxyl, alkyl or hydroxyalkyl, Y′ is hydroxyl or carboxyl; Q′ is alkyl or carboxyalkyl. Preferably, in formulae (I-a) and (I-b), R₂ is hydrogen, hydroxyl, carboxyl or hydroxymethyl and Q and Q′ are methyl or carboxymethyl.

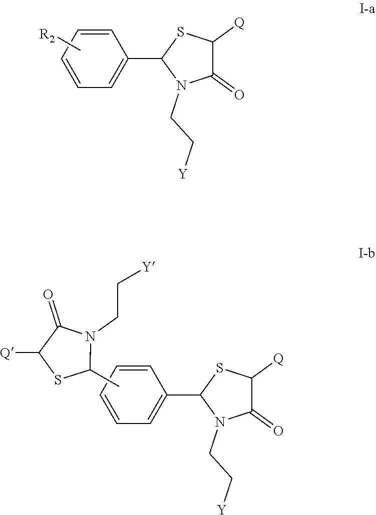

Monomers according to the first variant are very particularly the following compounds (1) to (8):

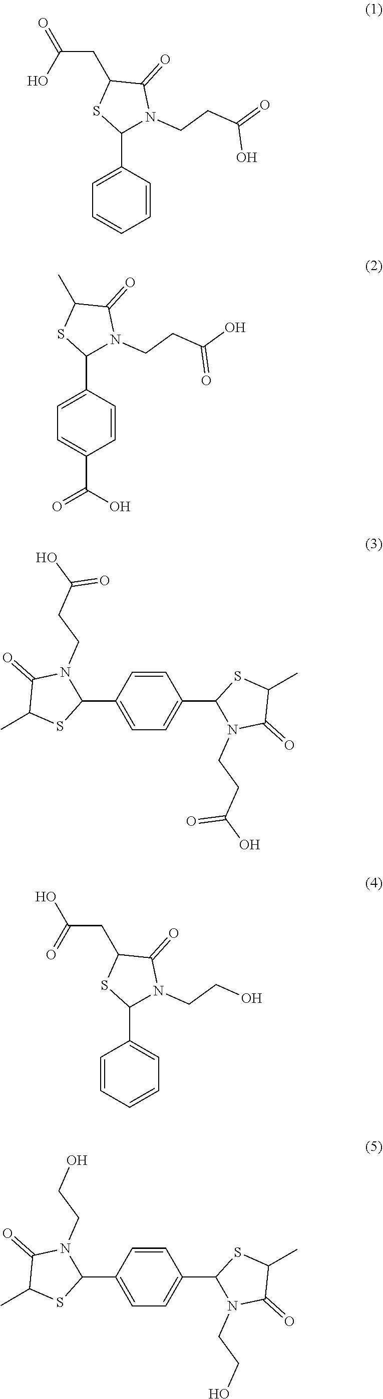

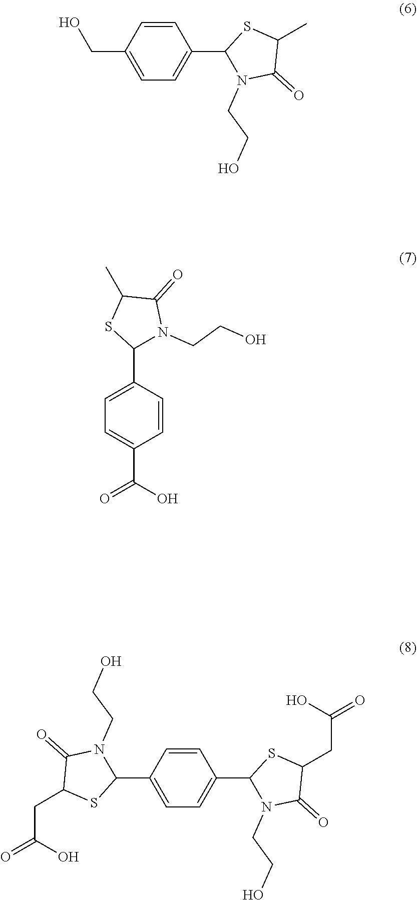 [SMILES: CC1SC(c2ccc(CO)cc2)N(CCO)C1=O ; CC1SC(c2ccc(C(=O)O)cc2)N(CCO)C1=O ; O=C(O)CC1SC(c2ccc(C3SC(CC(=O)O)C(=O)N3CCO)cc2)N(CCO)C1=O]

According to a first embodiment of the first variant, the monomer is of formula (I-a) in which R₂ is hydrogen, Y is hydroxyl or carboxyl and Q is carboxyalkyl, preferentially carboxymethyl. The monomers which satisfy this first embodiment are preferentially the compounds (4) and (1).

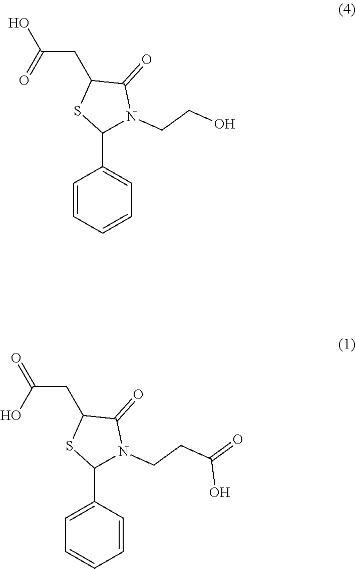 [SMILES: O=C(O)CC1SC(c2ccccc2)N(CCO)C1=O ; O=C(O)CCN1C(=O)C(CC(=O)O)SC1c1ccccc1]

According to a second embodiment of the first variant, the monomer is of formula (I-a) in which R₂ is carboxyl, Y is hydroxyl or carboxyl, Q is alkyl, in particular methyl. The monomers which satisfy this second embodiment are preferentially the compounds (7) and (2).

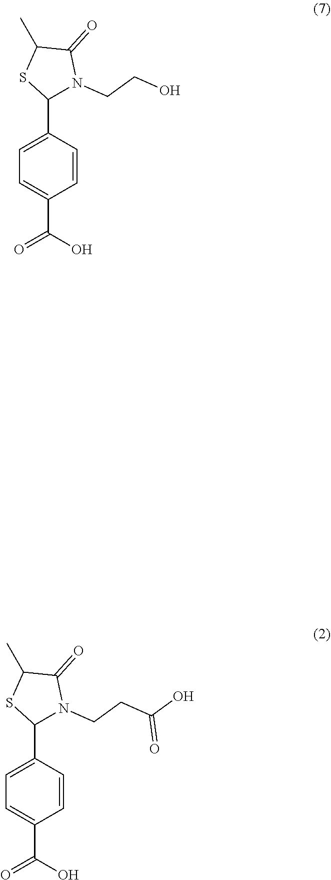 [SMILES: CC1SC(c2ccc(C(=O)O)cc2)N(CCO)C1=O ; CC1SC(c2ccc(C(=O)O)cc2)N(CCC(=O)O)C1=O]

According to a third embodiment of the first variant, the monomer is of formula (I-a) in which R₂ is hydroxyalkyl, preferentially hydroxymethyl, Y is hydroxyl or carboxyl, Q is alkyl, in particular methyl. A monomer which satisfies this third embodiment is preferentially compound (6).

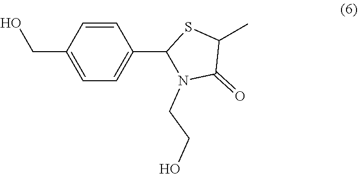 [SMILES: CC1SC(c2ccc(CO)cc2)N(CCO)C1=O]

According to a fourth embodiment of the first variant, the monomer is of formula (I-b) in which Y and Y′ are hydroxyl or carboxyl, Q and Q′ are alkyl or carboxyalkyl, in particular methyl or carboxymethyl. The monomers which satisfy this fourth embodiment are preferentially the compounds (3) and (5).

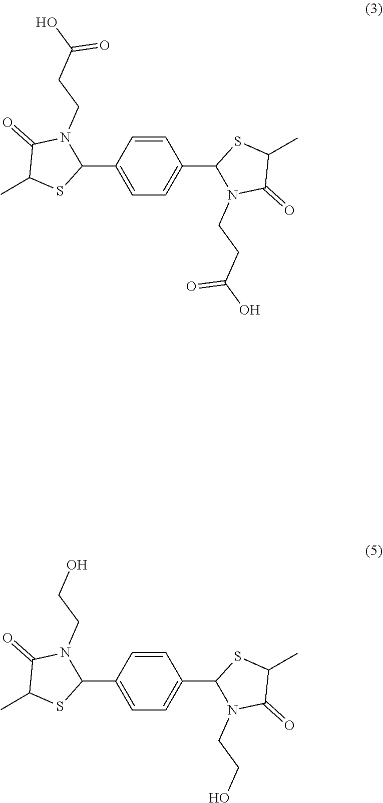

According to a fifth embodiment of the first variant, the monomer is of formula (I-b) in which Y and Y′ are hydroxyl or carboxyl, Q and Q′ are carboxyalkyl, in particular carboxymethyl. A monomer which satisfies this fifth embodiment is preferentially compound (8).

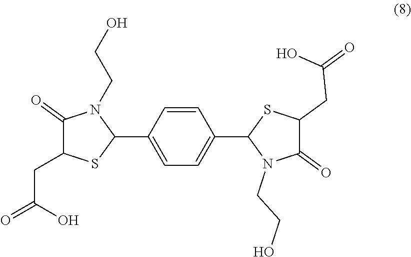 [SMILES: O=C(O)CC1SC(c2ccc(C3SC(CC(=O)O)C(=O)N3CCO)cc2)N(CCO)C1=O]

According to a second variant of the invention, the monomer is of formula (I-c) in which Y is hydroxyl or carboxyl, Q is alkyl or carboxyalkyl, Y′ is hydroxyl or carboxyl; Q′ is alkyl or carboxyalkyl, x is an integer ranging from 0 to 2.

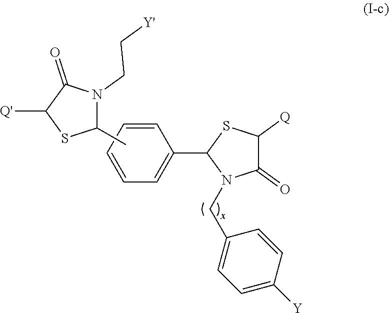

In formula (I-c), x can be equal to 0, 1 or 2. In formula (I-c), Q and Q′ are preferentially methyl. A monomer according to the second variant is very particularly compound (12).

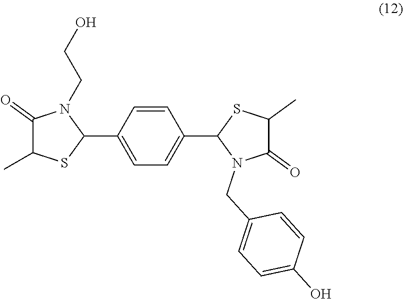 [SMILES: CC1SC(c2ccc(C3SC(C)C(=O)N3Cc3ccc(O)cc3)cc2)N(CCO)C1=O]

According to a third variant of the invention, the tire is of formula (II-1) in which Q is alkyl or carboxyalkyl, R₃ is hydrogen, hydroxyl, carboxyl, alkyl or hydroxyalkyl and A₁ is an alkanediyl or an alkanediyl interrupted by one or more oxygen atoms.

As monomers satisfying formula (II-1), mention may be made of the compounds (13) to (18).

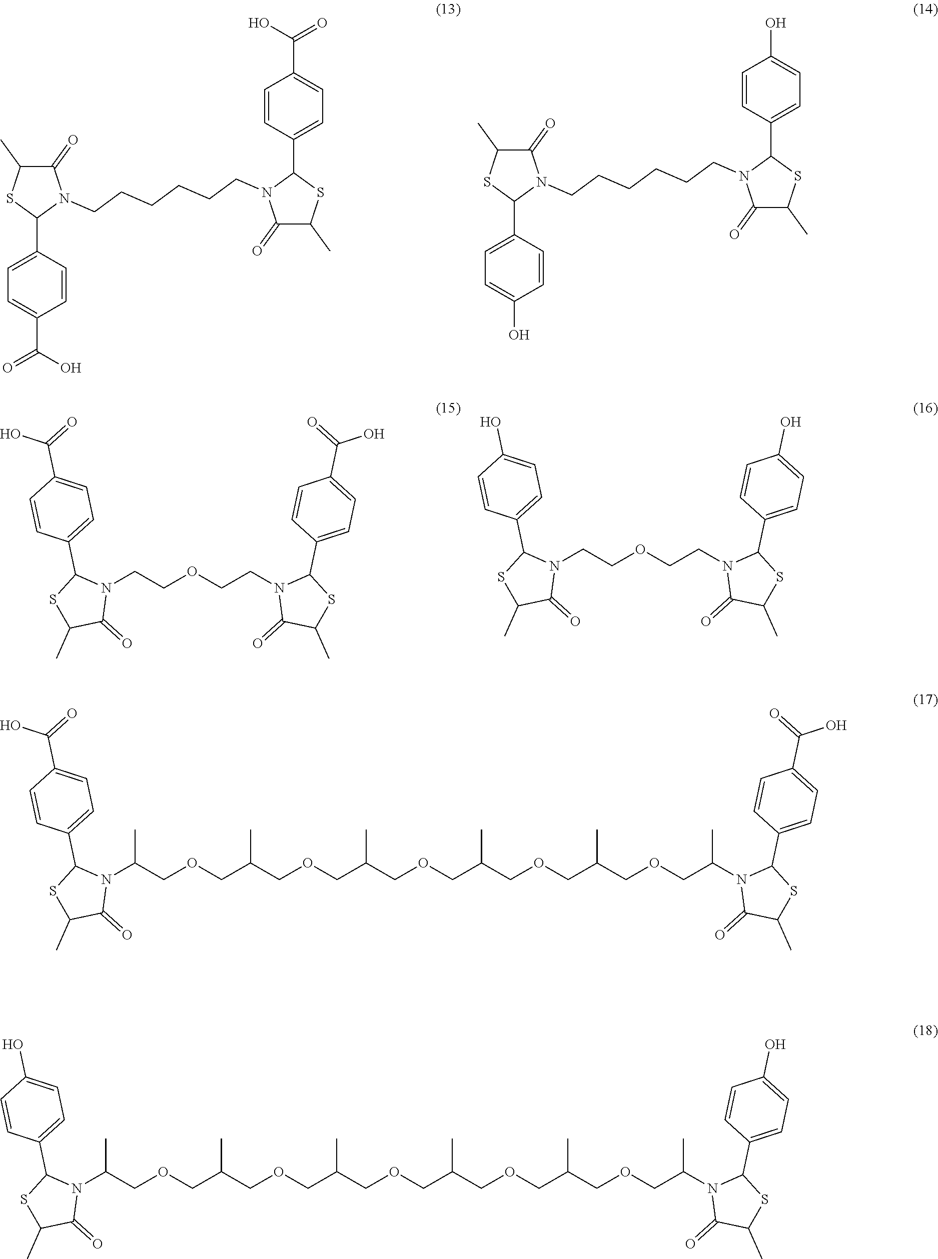

The monomer in accordance with the invention is typically prepared by a process, another subject of the invention, which comprises the reaction of an aldehyde of formula (II), a compound (III) and a compound of formula (IV)

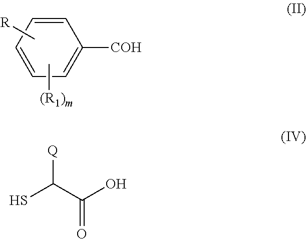

the compound (III) being of formula Y-A-NH₂ or of formula H₂N-A₁-NH₂,
in which Y is hydroxyl or carboxyl, Q is alkyl or carboxyalkyl, A is an ethanediyl, A₁ is an alkanediyl or an alkanediyl interrupted by one or more oxygen atoms, R₁ is alkyl, m is an integer ranging from 0 to 4, R is hydrogen, hydroxyl, carboxyl, alkyl, hydroxyalkyl, or an aldehyde function. One of the symbols Y, Q and R contains a hydroxyl (OH) or carboxyl (COOH) function. When R represents an aldehyde function, the process generally leads to a monomer of formula (I-b). The carbon group A₁ of compound (III) is of the same chemical structure as its counterpart in the monomers in accordance with the invention.

The monomers can be prepared according to reaction schemes 1 to 4.

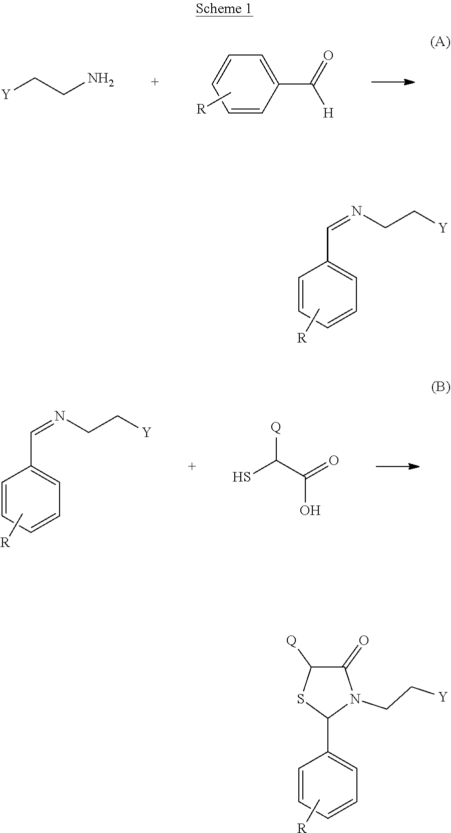

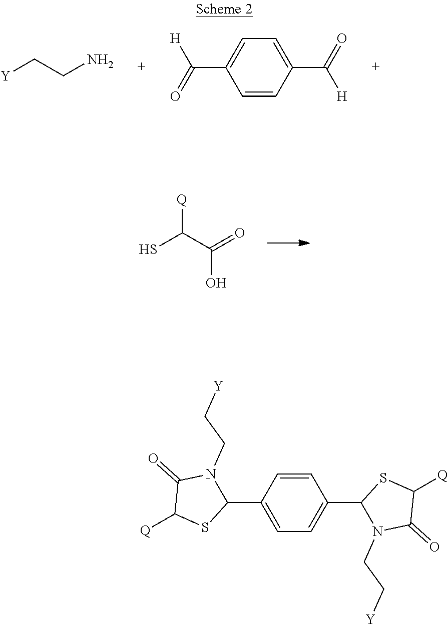

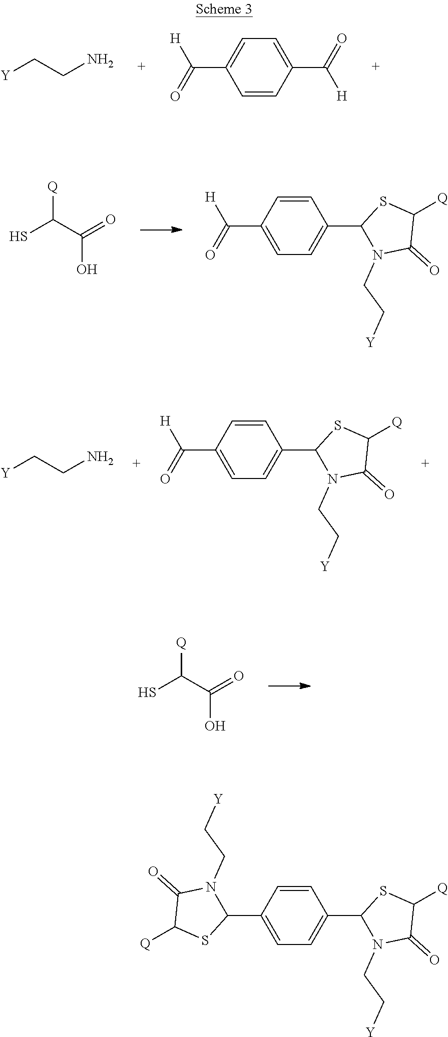

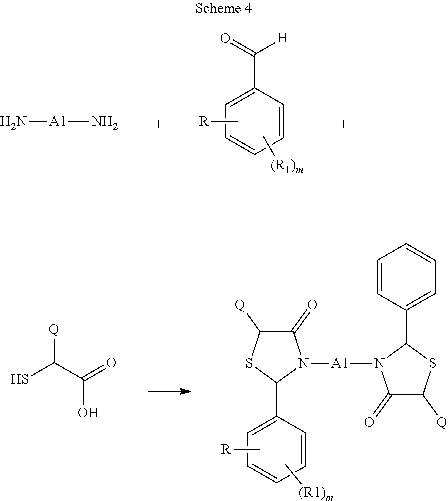

For example, monomers according to the first embodiment of the first variant can be prepared by a process involving one or other of the following reactions:

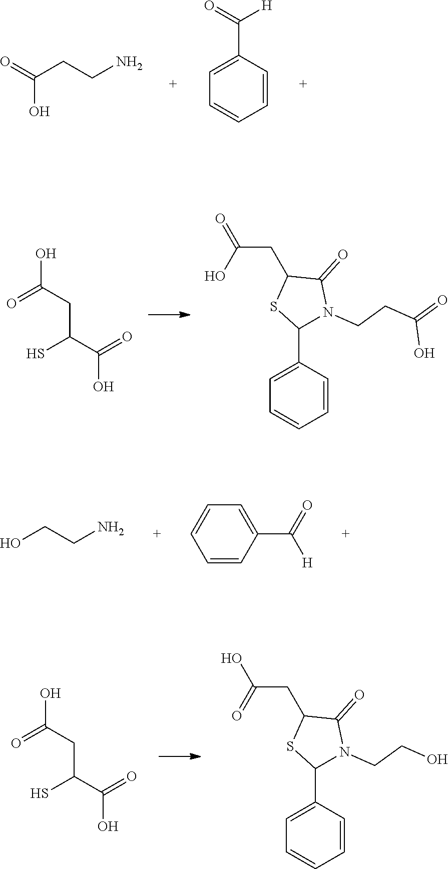

For example, monomers according to the second embodiment of the first variant can be prepared by a process involving one or other of the following reactions:

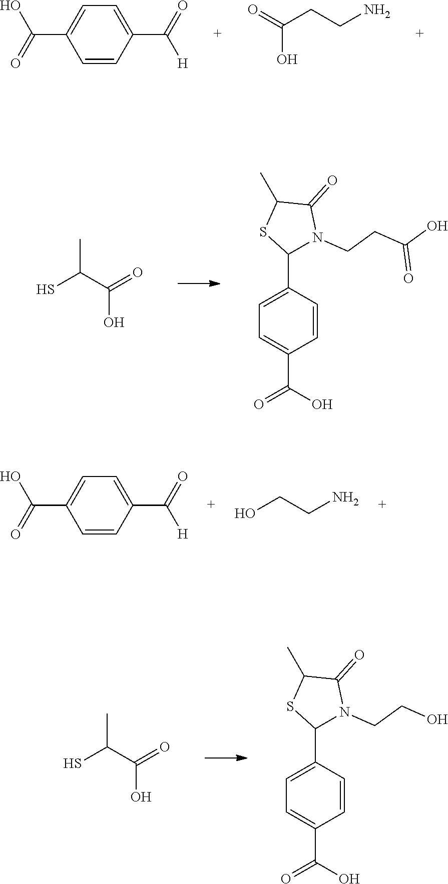

For example, monomers according to the third embodiment of the first variant can be prepared by a process involving the following reaction:

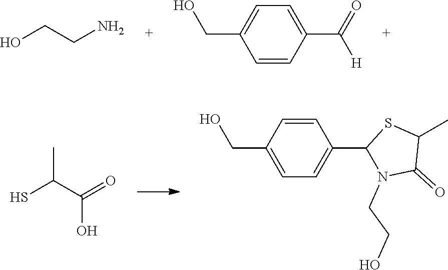

For example, monomers according to the fourth embodiment of the first variant can be prepared by a process involving one or other of the following reactions:

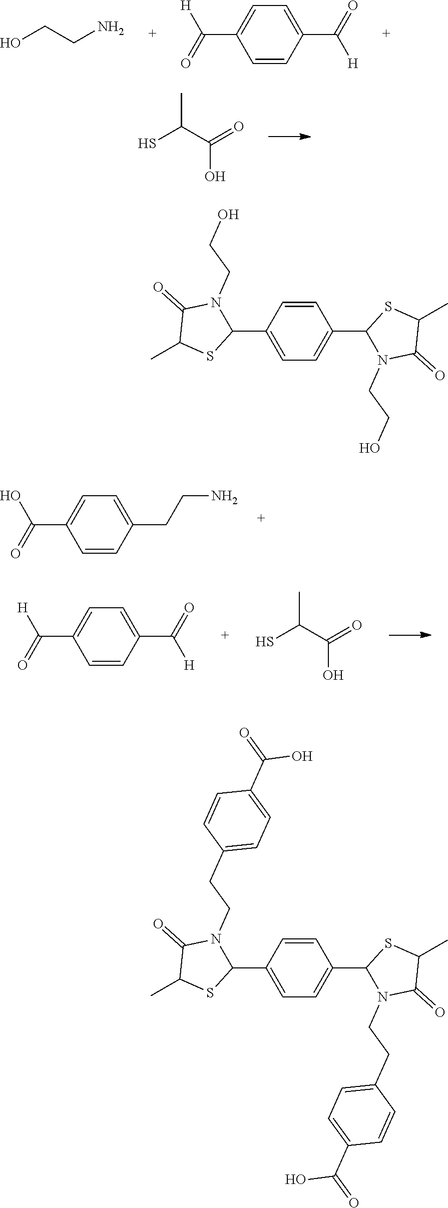

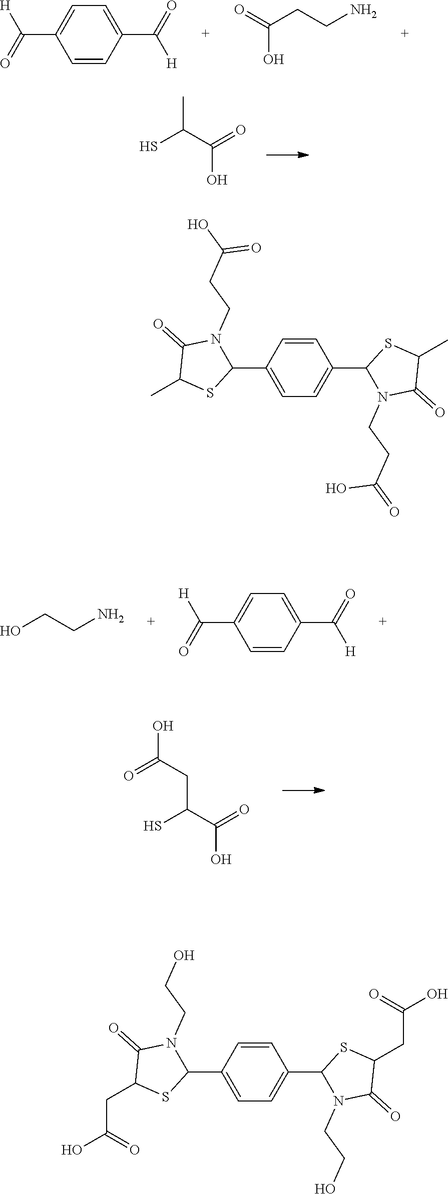

For example, monomers according to the second variant can be prepared by a process involving the following reaction scheme:

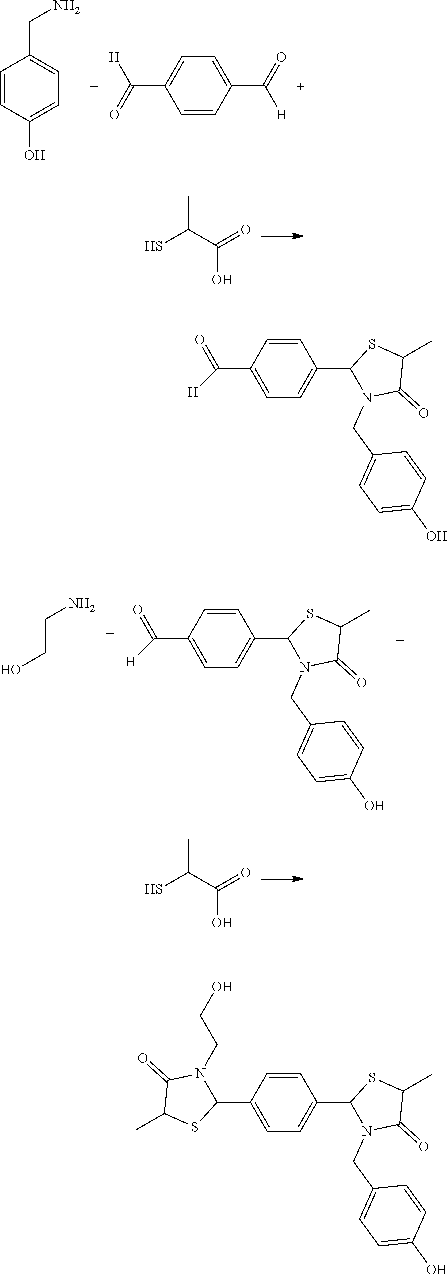

For example, monomers according to the third variant can be prepared by a process involving the following reaction scheme:

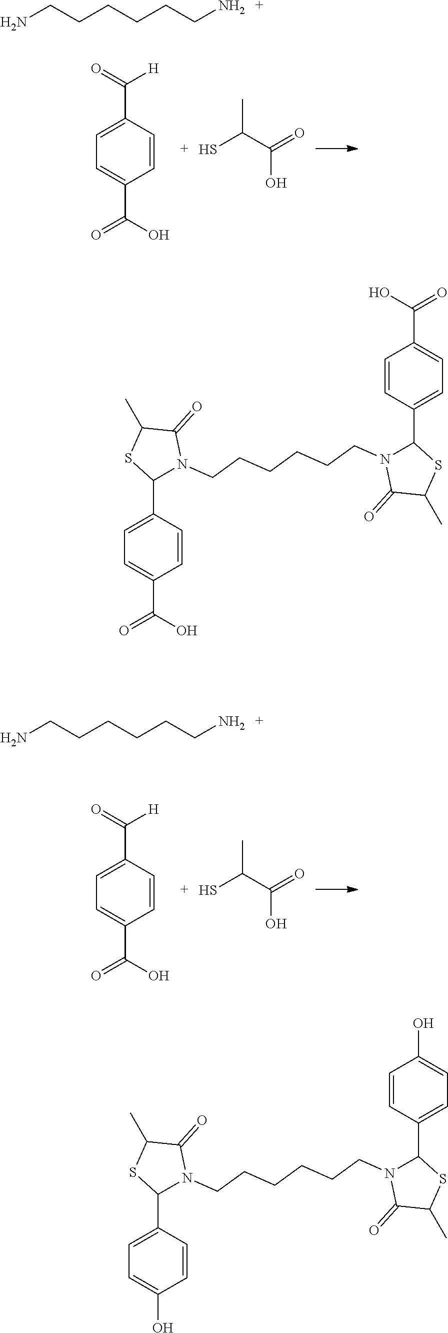

The reaction conditions for synthesizing the monomer are adapted by those skilled in the art according to the reaction scheme involved and taking into account the solubility of each of the substrates and the stoichiometry of the reaction.

The monomers in accordance with the invention are used in processes for synthesizing, respectively, polyamide, polyester and polyurethane polycondensates. In the processes for synthesizing the polycondensates, the two reactive functions of the monomer in accordance with the invention, which are selected from hydroxyl and carboxyl, can respectively be in the form of a hydroxylate (O⁻) or a carboxylate (COO⁻), in which case the monomer is in the form of a salt in the process for synthesizing the polycondensate. The two reactive functions of the monomer in accordance with the invention are those which take part in the polycondensation reaction with another monomer, the “second monomer”, to form a polycondensate. The second monomer is typically a compound which contains two functions which are reactive respectively with the two reactive functions of the monomer in accordance with the invention. The two functions of the second monomer are covalently linked to each other via a spacer, a divalent, aromatic or aliphatic hydrocarbon chain, optionally interrupted by one or more heteroatoms such as oxygen, nitrogen or sulfur. The carbon number of the spacer can vary to a large extent. It preferentially varies from 4 to 20. As divalent hydrocarbon chain, mention may be made of alkanediyls, arenediyls, alkylarenediyls. The second monomer can also be a monomer according to the invention and be of formula (I-a), (I-b), (I-c) or (II-1).

When the monomer in accordance with the invention has two carboxyl functions as reactive function, it reacts according to a first process for synthesizing polycondensates with a second monomer which contains two functions selected from amine and isocyanate, to form a polyamide. When the monomer is of formula (I-a), two of the symbols Y, Q and R₂ are carboxyls; when the monomer is of formula (I-b), two of the symbols Y, Q, Y′ and Q′ are carboxyls; when the monomer is of formula (II-1), two of the symbols R₃ and Q are carboxyls. In the first process, the compounds of formulae (1), (2), (3), (8), (13), (15) and (17) can be used. The second monomer can be a diamine or a diisocyanate. The two monomers react, in an equivalent manner, in the form of their salt, the monomer in accordance with the invention being in the form of a carboxylate salt, the second monomer being a diamine in the form of a hydrohalate salt.

Suitable as second monomer are, for example, hexamethylenediamine or 1,6-diaminohexane, 1,12-diaminododecane, diphenylmethane diisocyanate (“MDI”), toluene diisocyanate (“TDI”), naphthalene diisocyanate (“NDI”), 3,3′-bitoluene diisocyanate (“TODI”), para-phenylene diisocyanate (“PPDI”), 1,4-tetramethylene diisocyanate, 1,6-hexane diisocyanate (“HDI”), 1,4-bis(isocyanatomethyl)cyclohexane, 1,3-bis(isocyanatomethyl)cyclohexane, 1,3-bis(isocyanatomethyl)benzene, 1,4-bis(isocyanatomethyl)benzene, isophorone diisocyanate (“IPDI”), bis(4-isocyanatocyclohexyl)methane diisocyanate (“H12MDI”) and 4,4′-dicyclohexylmethane diisocyanate (“H13MDI”).

When the monomer in accordance with the invention has a carboxyl function and a hydroxyl function as reactive function, it reacts according to a second process for synthesizing polycondensates with a second monomer which itself also contains a carboxyl function and a hydroxyl function, to form a polyester. When the monomer is of formula (I-a), two of the symbols Y, Q and R₂ are carboxyl and hydroxyl, respectively; when the monomer is of formula (I-b), two of the symbols Y, Q, Y′ and Q′ are carboxyl and hydroxyl, respectively. In the second process, the compounds of formulae (4), (7) and (8) can be used. When the monomer is of formula (II-1), two of the symbols Q and R₃ are carboxyl and hydroxyl, respectively.

When the monomer in accordance with the invention, such as the compounds of formulae (5), (6), (8), (14), (16) and (18), has two hydroxyl functions as reactive function, it reacts according to a third process for synthesizing polycondensates with a second monomer which contains two carboxyl functions, to form a polyester. Suitable as second monomer is any aliphatic or aromatic diacid, such as 1,4-butanedioic acid, terephthalic acid, isophthalic acid or the compounds of formulae (1), (2), (3), (8), (13), (15) and (17).

Alternatively, to form a polyester according to the third process, the monomer in accordance with the invention, such as the compounds of formulae (1), (2), (3), (8), (13), (15) and (17), has two carboxyl functions as reactive function, and the second monomer contains two hydroxyl functions. Suitable as second monomer is any aliphatic or aromatic diol, such as 1,4-butanediol, hydroquinone bis(2-hydroxyethyl) ether (HQEE), resorcinol bis(2-hydroxyethyl) ether (HER) or else the compounds of formulae (5), (6) and (8).

When the monomer in accordance with the invention, such as the compounds of formulae (5), (6), (8), (14), (16) and (18), has two hydroxyl functions as reactive function, it reacts according to a fourth process for synthesizing polycondensates with a second monomer which contains two isocyanate functions, to form a polyurethane. The second monomer is typically a diisocyanate as defined in the first process for synthesizing polycondensates.

In the processes for synthesizing polycondensates in accordance with the invention, the second monomer can be partly or totally replaced by a prepolymer which contains two reactive functions identical to that of the second monomer. The term prepolymer is used according to the meaning given by IUPAC in the Gold Book. Suitable as prepolymer are, for example, the following oligomers, p being equal to or greater than 1:

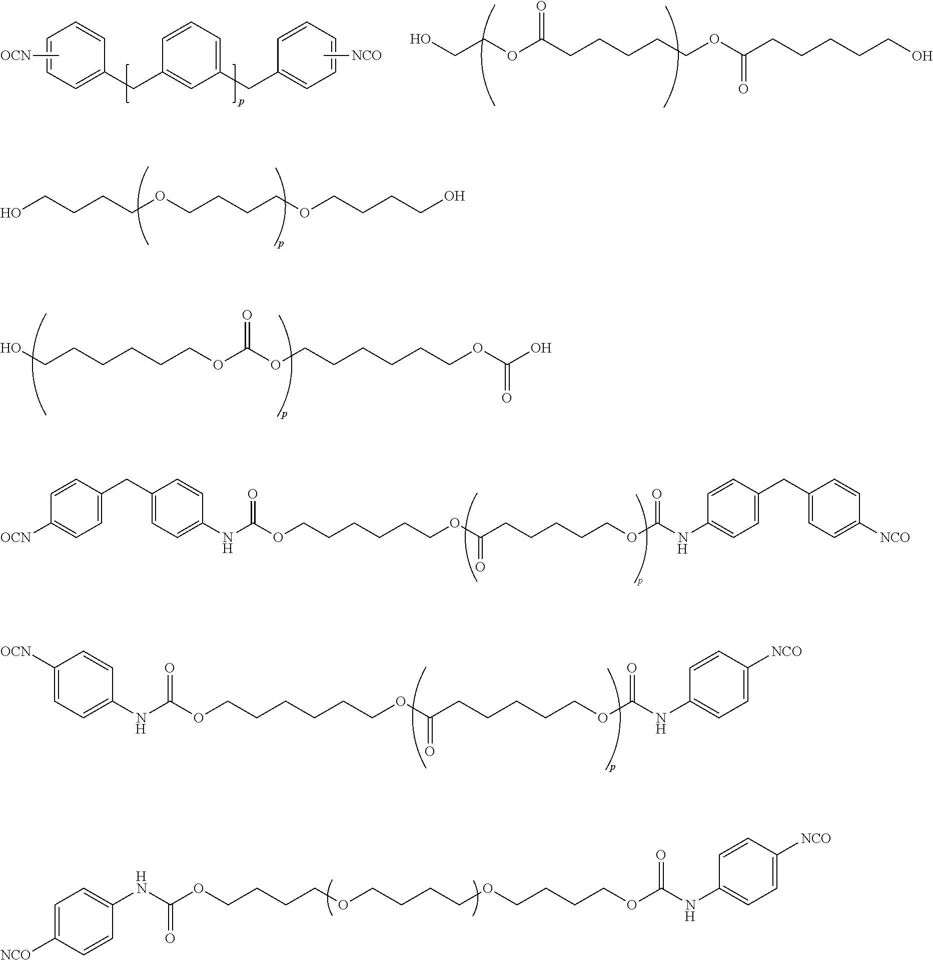

The 3 isocyanate prepolymers are called MDI prepolymer for the first and PPDI for the other two.

The reaction conditions for forming the polycondensates according to the processes in accordance with the invention are those commonly used in the synthesis of polyamides and are well known to those skilled in the art. Those skilled in the art will adapt them according to the solubility of the reagents and the stoichiometry of the reaction.

The polycondensates that can be obtained by the processes for synthesizing polycondensates in accordance with the invention are polycondensates containing monomer units comprising 4-thiazolidinone units. Thus, a polycondensate that can be obtained by the first polycondensate synthesis process according to the invention is a polyamide which contains monomer units comprising 4-thiazolidinone units; a polycondensate that can be obtained by the second or the third polycondensate synthesis process according to the invention is a polyester which contains monomer units comprising 4-thiazolidinone units; a polycondensate that can be obtained by the fourth polycondensate synthesis process according to the invention is a polyurethane which contains monomer units comprising 4-thiazolidinone units.

The polyamide, polyester and polyurethane polycondensates according to the invention have improved thermal properties compared to their respective polyamide, polyester and polyurethane counterparts which do not contain 4-thiazolidinone units.

In summary, the invention is advantageously implemented according to any one of the following embodiments 1 to 24:

Embodiment 1: Monomer which contains a 4-thiazolidinone unit connected to a benzene nucleus and which contains two functions selected from hydroxyl and carboxyl, which monomer is of formula (I-a) or (I-b) in which Y is hydroxyl or carboxyl, Q is alkyl or carboxyalkyl, R₂ is hydrogen, hydroxyl, carboxyl, alkyl or hydroxyalkyl, Y′ is hydroxyl or carboxyl; Q′ is alkyl or carboxyalkyl,

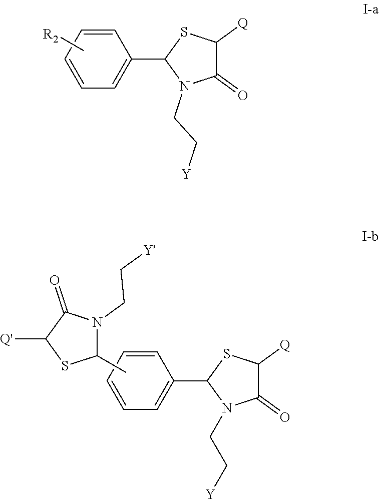

or of formula (I-c) in which Y is hydroxyl or carboxyl, Q is alkyl or carboxyalkyl, Y′ is hydroxyl or carboxyl; Q′ is alkyl or carboxyalkyl, x is an integer ranging from 0 to 2,

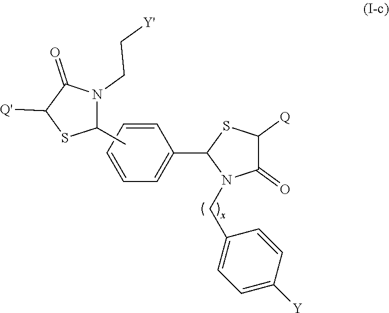

or else of formula (II-1) in which Q is alkyl or carboxyalkyl, R₁ is alkyl, R₃ is hydrogen, hydroxyl, carboxyl, alkyl or hydroxyalkyl and A₁, a divalent carbon group which contains carbon atoms, is an alkanediyl or an alkanediyl interrupted by one or more oxygen atoms, m is an integer ranging from 0 to 4.

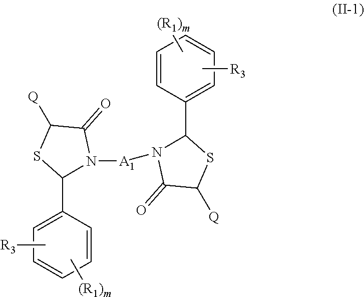

Embodiment 2: Monomer according to Embodiment 1, wherein R₂ and R₃ are para or meta relative to the thiazolidinone ring when they are different from a hydrogen atom.

Embodiment 3: Monomer according to Embodiment 1 or 2, wherein R₂ and R₃ are para relative to the thiazolidinone ring when they are different from a hydrogen atom.

Embodiment 4: Monomer according to any one of Embodiments 1 to 3, wherein m is equal to 0.

Embodiment 5: Monomer according to any one of Embodiments 1 to 4, wherein the alkyls represented by Q and Q′ are methyl.

Embodiment 6: Monomer according to any one of Embodiments 1 to 5, wherein the carboxyalkyls represented by Q and Q′ are carboxymethyl.

Embodiment 7: Monomer according to any one of Embodiments 1 to 6, wherein the hydroxyalkyls represented by R₂ and R₃ are hydroxymethyl.

Embodiment 8: Monomer according to any one of Embodiments 1 to 7, wherein the alkyls represented by R_{1,} R_{2,} R₃ are alkyls having 1 to 3 carbon atoms.

Embodiment 9: Monomer according to any one of Embodiments 1 to 8, wherein the alkyls represented by R₁, R₂, R₃ are methyl.

Embodiment 10: Monomer according to any one of Embodiments 1 to 9, wherein Q and Q′ are alkyl or carboxyalkyl.

Embodiment 11: Monomer according to any one of Embodiments 1 to 10, wherein A₁ contains 2 to carbon atoms.

Embodiment 12: Monomer according to any one of Embodiments 1 to 11, wherein A₁ contains at least 4 carbon atoms.

Embodiment 13: Monomer according to any one of Embodiments 1 to 12, which monomer is of formula (I-a) or (I-b) in which Y is hydroxyl or carboxyl, Q is alkyl or carboxyalkyl, R₂ is hydrogen, hydroxyl, carboxyl, alkyl or hydroxyalkyl, Y′ is hydroxyl or carboxyl; Q′ is alkyl or carboxyalkyl.

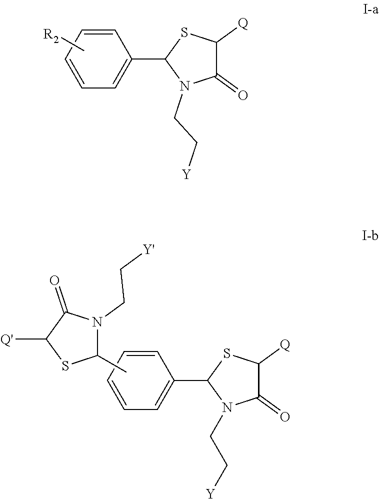

Embodiment 14: Monomer according to any one of Embodiments 1 to 10, which monomer is of formula (I-c) in which Y is hydroxyl or carboxyl, Q is alkyl or carboxyalkyl, Y′ is hydroxyl or carboxyl; Q′ is alkyl or carboxyalkyl, x is an integer ranging from 0 to 2.

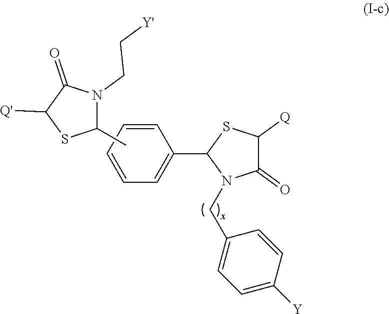

Embodiment 15: Monomer according to any one of Embodiments 1 to 14, wherein R₂ is hydrogen, hydroxyl, carboxyl, hydroxyalkyl.

Embodiment 16: Monomer according to any one of Embodiments 1 to 11, which monomer is of formula (II-1) in which Q is alkyl or carboxyalkyl, R₃ is hydrogen, hydroxyl, carboxyl, alkyl or hydroxyalkyl and A₁ is an alkanediyl or an alkanediyl interrupted by one or more oxygen atoms.

Embodiment 17: Monomer according to any one of Embodiments 1 to 11 or 16, wherein R₃ is hydroxyl or carboxyl.

Embodiment 18: Process for synthesizing a monomer defined in any one of Embodiments 1 to 17 which comprises the reaction of an aldehyde of formula (II), a compound (III) and a compound of formula (IV)

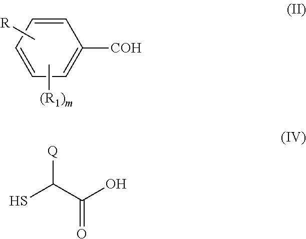

the compound (III) being of formula Y-A-NH₂ or of formula H₂N-A₁-NH₂,
in which Y is hydroxyl or carboxyl, Q is hydrogen, alkyl or carboxyalkyl, A is ethanediyl, Al is an alkanediyl or an alkanediyl interrupted by one or more oxygen atoms, R₁ is alkyl, m is an integer ranging from 0 to 4, R is hydrogen, hydroxyl, carboxyl, alkyl, hydroxyalkyl, or an aldehyde function.

Embodiment 19: Process for synthesizing a polyamide which comprises the reaction of a monomer defined in any one of Embodiments 1 to 17 with a second monomer containing two functions selected from amine and isocyanate, the monomer of formula defined in any one of Embodiments 1 to 17 containing two carboxyl functions.

Embodiment 20: Process for synthesizing a polyester which comprises the reaction of a monomer defined in any one of Embodiments 1 to 17 with a second monomer, both monomers each containing a carboxyl function and a hydroxyl function or alternatively one of the two monomers containing two hydroxyl functions and the other containing two carboxyl functions.

Embodiment 21: Process for synthesizing a polyester which comprises the reaction of a monomer defined in any one of Embodiments 1 to 17 with a second monomer, one of the two monomers containing two hydroxyl functions and the other containing two carboxyl functions.

Embodiment 22: Process for synthesizing a polyurethane which comprises the reaction of a monomer defined in any one of Embodiments 1 to 17 with a second monomer containing two isocyanate functions, the monomer of formula defined in any one of Embodiments 1 to 17 containing two hydroxyl functions.

Embodiment 23: Process according to any one of Embodiments 19 to 22, wherein the second monomer is partly or totally replaced by a prepolymer which contains two reactive functions identical to that of the second monomer.

Embodiment 24: Polycondensate capable of being obtained by a process defined in any one of Embodiments 19 to 22, the polycondensate being a polyamide, a polyester or a polyurethane and containing monomer units comprising 4-thiazolidinone units.

The abovementioned characteristics of the present invention, and also others, will be better understood on reading the following description of several exemplary embodiments of the invention, which are given as nonlimiting illustrations.

### EXAMPLES

Thermal characterizations by differential scanning calorimetry (DSC) were carried out using a Mettler Toledo DSC3 apparatus. The sample (about 10 mg) is weighed and sealed in a 40 μL aluminium crucible. The crucible is pierced with a fine needle just before the measurement. Dry nitrogen is used as purge gas with a flow of 30 mL min⁻¹. Two successive cycles of heating and cooling are carried out identically. For each cycle, the sample and also an empty reference crucible are heated from a minimum temperature to a maximum temperature at 10° C. min⁻¹. The temperature is maintained for 2 minutes at the maximum temperature, then cooled from the maximum temperature to the minimum temperature at 10° C. min⁻¹. Unless otherwise indicated, the glass transition temperature (Tg) and the melting temperature (Tm) are measured in the second cycle. If appropriate, in the event of mass loss observed during the second cycle, a third cycle is carried out under the same conditions as the second cycle. The glass transition temperature (Tg) corresponds to the temperature which is the midpoint temperature (T mg) as defined in the standard ASTM D3418-99. The melting temperature (Tm) corresponds to the tip of the melting peak.

The dynamic mechanical analysis (DMA) in tension/compression was carried out on a test specimen 24 mm long, 3 mm thick and 5 mm wide, with a Metravib DMA40+ apparatus at a frequency of 10 Hz with a temperature sweep from −120° C. to 180° C. with a ramp of 2° C./min, unless otherwise indicated.

DMA analysis measures the glass transition temperature and the Young's modulus (E′) at a given temperature. The glass transition temperature measured by DMA corresponds to the temperature at which the loss modulus (E″) reaches its maximum.

All of the syntheses are carried out under a nitrogen atmosphere, unless otherwise indicated.

### Monomer According to the Invention, Compound (6): Example 1

### Synthesis of 3-(2-hydroxyethyl)-2-(4-hydroxymethylphenyl)-5-methylthiazolidin-4-one by reaction of 2-mercaptopropionic acid, 2-aminoethanol and 4-(hydroxymethyl)benzaldehyde

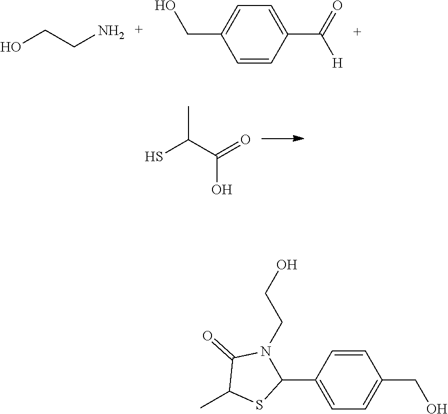

A 100 ml four-necked flask, dried beforehand under vacuum at more than 100° C. and equipped with a condenser, bubbler, thermometer and magnetic stirrer bar, is charged with 4.085 g (i.e. 30 mmol) of 4-(hydroxymethyl)benzaldehyde (99%, Fluorochem, #222581) followed by 1.83 g (i.e. 30 mmol) of ethanolamine. Exothermicity to around 40° C. is observed and the whole becomes rapidly soluble with stirring. To finish, 3.35 g (i.e. 30 mmol) of 95% 2-mercaptopropionic acid are added. The reaction medium is heated to 75° C. and stirred for 18 hours under a gentle stream of nitrogen. The monomer is purified by chromatography using a mixture of ethyl acetate and heptane (4:1), followed by ethyl acetate and methanol (95:5). The structure of the product is confirmed by nuclear magnetic resonance (NMR) analysis.

¹H RMN (600 MHz, DMSO-d6) δ 7.54-7.11 (m, 4H), 5.81 (s, 1H), 5.20 (t, J=5.7 Hz, 1H), 4.74 (s, 1H), 4.48 (t, J=5.0 Hz, 2H), 3.99 (dd, J=27.0, 7.1 Hz, 1H), 3.53 (d, J=13.8 Hz, 1H), 3.40 (s, 1H), 3.29 (s, 1H), 2.59 (d, J=13.7 Hz, 1H), 1.45 (dd, J=25.5, 7.0 Hz, 3H).

### Monomer According to the Invention, Compound (4): Example 2

### Synthesis of [3-(2-hydroxyethyl)-4-oxo-2-phenylthiazolidin-5-yl]acetic acid by reaction of mercaptosuccinic acid, ethanolamine and benzaldehyde

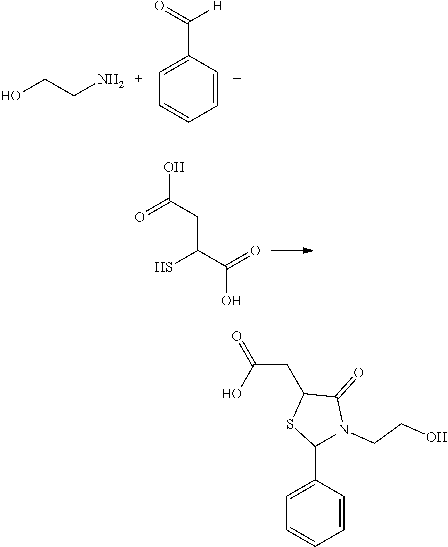

A 100 ml four-necked flask, dried beforehand under vacuum at more than 100° C. and equipped with a condenser, bubbler, thermometer and magnetic stirrer bar, is charged with 6.96 g (i.e. 65.6 mmol) of benzaldehyde followed by 10 ml of anhydrous methanol. With stirring, 4.0 g (i.e. 65.6 mmol) of ethanolamine are added and exothermicity to around 40° C. is observed. The reaction mixture is cooled to ambient temperature using an ice bath and 9.85 g (i.e. 65.6 mmol) of mercaptosuccinic acid (99%, Alrich #88460) are added to finish. The reaction medium is stirred at ambient temperature for 65 hours under a gentle stream of nitrogen. The methanol is evaporated and a viscous (honey) light yellow liquid is obtained. After purification of 9.8 g of crude product on silica gel (eluent: pentane/ethyl acetate (70/30 by volume), then ethyl acetate), 5.2 g of white solid are isolated.

¹H RMN (600 MHz, DMSO-d6) δ 12.54 (s, 1H), 7.57-7.20 (m, 5H), 5.84 (s, 1H), 4.75 (t, J=5.6 Hz, 1H), 4.38-4.08 (m, 1H), 3.55 (dt, J=13.7, 6.0 Hz, 1H), 3.42 (dd, J=11.2, 5.8 Hz, 1H), 3.10 (dd, J=17.1, 3.6 Hz, 1H), 2.67 (dd, J=17.1, 10.1 Hz, 1H), 2.58 (dt, J=13.7, 6.2 Hz, 1H).

### Monomer According to the Invention, Compound (5): Example 3

### Synthesis of 2,2′-(1,4-phenylene)bis[3-(2-hydroxyethyl)-5-methyl-1,3-thiazolidin-4-one] by reaction of terephthalaldehyde, ethanolamine and 2-mercaptopropionic acid

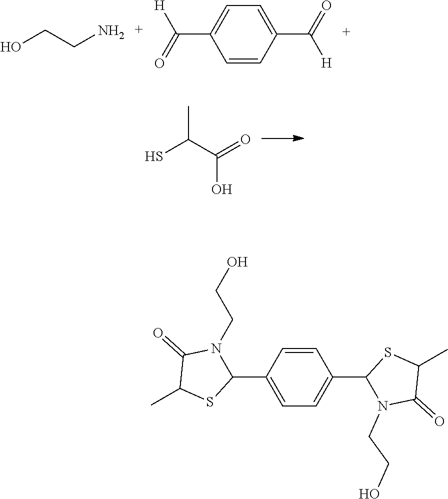

A 100 ml four-necked flask, dried beforehand under vacuum at more than 100° C. and equipped with a condenser, bubbler, thermometer and magnetic stirrer bar, is charged with 13.4 g (i.e. 100 mmol) of terephthalaldehyde followed by 44.69 g (i.e. 400 mmol) of 95% 2-mercaptopropionic acid. Exothermicity to around 40° C. is observed and the mixture is stirred at ambient temperature until the solid dissolves. 12.22 g (i.e. 200 mmol) of ethanolamine are then added in one go; exothermicity is observed to around 105° C. The reaction medium is stirred for 24 hours at ambient temperature under a gentle stream of nitrogen. After 24 hours of reaction, a 10 g aliquot is taken which is dissolved in 300 ml of butanone (ethyl methyl ketone) at 40° C. The organic phase is washed with 3×300 ml of 0.6M NaOH (pH 9) and 3×300 ml of 20% NaCl (aqueous solutions). The aqueous phase is re-extracted with 3×300 ml of butanone. The combined organic phases are dried over Na₂SO₄, filtered and evaporated. 7 g of a viscous yellow liquid are obtained, this being purified on silica gel using an ethyl acetate and heptane solvent mixture. A white solid is isolated after purification.

¹H RMN (600 MHz, DMSO-d6) δ 7.50-7.27 (m, 2H), 6.00-5.74 (m, 1H), 4.78 (t, J=5.5 Hz, 1H), 4.00 (qd, J=7.1, 6.1 Hz, 1H), 3.58 (dt, J=13.7, 5.7 Hz, 1H), 3.45 (dd, J=11.1, 5.9 Hz, 1H), 3.35 (dd, J=11.0, 5.6 Hz, 1H), 2.60 (dt, J=13.6, 6.4 Hz, 1H), 1.56-1.41 (m, 3H).

### Monomer According to the Invention, Compound (8): Example 4

### Synthesis of 2-[2-[4-[5-(carboxymethyl)-3-(2-hydroxyethyl)-4-oxothiazolidin-2-yl]phenyl]-3-(2-hydroxyethyl)-4-oxothiazolidin-5-yl]acetic acid by reaction of mercaptosuccinic acid, ethanolamine and terephthalaldehyde

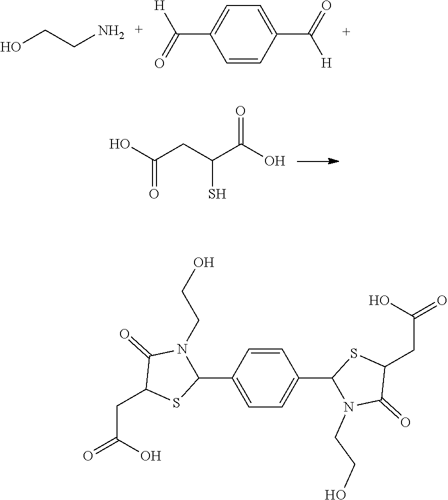

A 100 ml four-necked flask, dried beforehand under vacuum at more than 100° C. and equipped with a condenser, bubbler, thermometer and magnetic stirrer bar, is charged with 2.68 g (i.e. 20 mmol) of terephthalaldehyde, followed by 60 ml of anhydrous methanol. After dissolution with stirring at ambient temperature, 2.44 g (i.e. 40 mmol) of ethanolamine and 6 g (i.e. 40 mmol) of mercaptosuccinic acid are added. The reaction medium is stirred at reflux for 48 hours under a gentle stream of nitrogen. The methanol is evaporated and a viscous golden yellow liquid is obtained. The two monomers obtained (spot A less polar than spot B) are separated by preparative thin layer chromatography (TLC) in ethyl acetate and methanol (12:8). FTIR spectrum analysis does not show any significant difference between spot A and spot B. NMR analysis in DMSO-d6 shows that spot B (the most polar) corresponds to the expected monomer.

### Monomer According to the Invention, Compound (12): Example 5

### Synthesis of 3-[(4-hydroxyphenyl)methyl]-2-[4-[3-[(4-hydroxyphenyl)methyl]-5-methyl-4-oxothiazolidin-2-yl]phenyl]-5-methylthiazolidin-4-one by reaction of terephthalaldehyde, 4-hydroxybenzylamine and 2-mercaptopropionic acid

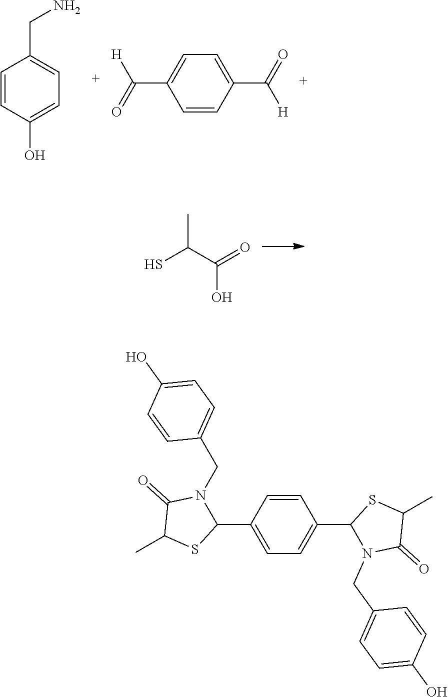

A 250 ml four-necked flask, dried beforehand under vacuum at more than 100° C. and equipped with a condenser, bubbler, thermometer and magnetic stirrer bar, is charged with 13.4 g (i.e. 100 mmol) of terephthalaldehyde, followed by 60 ml of anhydrous DMSO. The mixture is stirred at 50° C. to facilitate dissolution of the solid, then allowed to return to ambient temperature. A solution of 24.63 g (i.e. 200 mmol) of 4-hydroxybenzylamine in 40 ml anhydrous DMSO is prepared; to aid dissolution of the amine, the solution is heated to 50° C. The amine solution is added to the solution containing the aldehyde with stirring. To finish, 44.69 g (i.e. 400 mmol) of 95% 2-mercaptopropionic acid are added. Exothermicity to around 60° C. is observed. The reaction mixture is stirred at ambient temperature for 48 hours under a gentle stream of nitrogen. After 48 hours of reaction, a 40 ml aliquot portion is taken which is poured into 400 g of ice; the whole is mixed using a spatula and an ice-precipitate mixture is thus obtained. This mixture is placed on a Büchner filter. The mixture is rinsed with four 250-ml portions of iced water. The precipitate-ice mixture is placed in a 2 L beaker and 500 ml of acetone are added; this mixture is stirred until the solid has dissolved. This solution is placed in a dropping funnel and is slowly added to 5 litres of iced water with stirring. A white precipitate forms and is isolated by Büchner filtration. 43.2 g of a white solid are obtained, this being purified by silica gel chromatography using an ethyl acetate and heptane (1:1) solvent mixture.

¹H RMN (500 MHz, DMSO) δ 9.40 (s, 2H), 7.59-7.10 (m, 4H), 7.02-6.77 (m, 4H), 6.74-6.48 (m, 4H), 5.55-5.30 (m, 2H), 4.89-4.56 (m, 2H), 4.22 (d, J=7.0 Hz, 1H), 4.07 (dd, J=7.0, 2.7 Hz, 1H), 3.54 (dd, J=14.7, 9.9 Hz, 2H), 1.75-1.51 (m, 3H), 1.51-1.32 (m, 3H).

Compound (12) is also prepared according to the procedure described above, with the difference that 22.35 g of 95% 2-mercaptopropionic acid are used and that the reaction takes place at 75° C. Under these reaction conditions, it is not necessary to purify the product by silica gel chromatography.

### Monomer According to the Invention, Compound (3): Example 6

### Synthesis of 3-[2-[4-[3-(2-carboxyethyl)-5-methyl-4-oxothiazolidin-2-yl]phenyl]-5-methyl-4-oxothiazolidin-3-yl]propanoic acid by reaction of 2-mercaptopropionic acid, 13-alanine and terephthalaldehyde

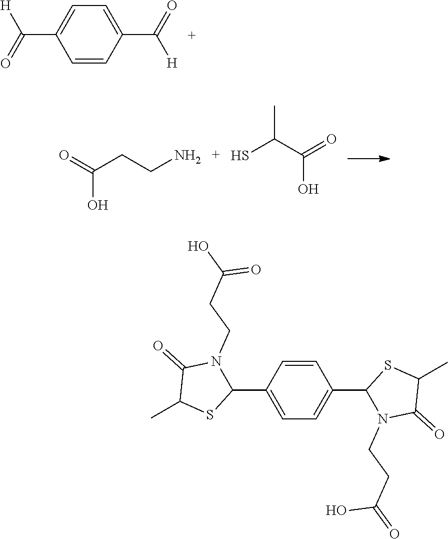

A 250 ml four-necked flask, dried beforehand under vacuum at more than 100° C. and equipped with a condenser, bubbler, thermometer and magnetic stirrer bar, is charged with 6.7 g of terephthalaldehyde (50 mmol), followed by 30 ml of DMSO with stirring at ambient temperature. The mixture is stirred at ambient temperature for 5 minutes to achieve partial dissolution of the aldehyde, to which 22.35 g of 95% 2-mercaptopropionic acid (200 mmol) (Sigma-Aldrich, W318000) are added rapidly and in one go. The reaction medium becomes clear with an increase in the temperature to around 42° C. After stirring the reaction medium for 2 minutes at ambient temperature, 8.91 g of 99% 13-alanine (100 mmol) (“BioExtra”, Sigma Aldrich 05160) are added rapidly and in one go. A total of 20 ml of DMSO is used to rinse the equipment used when introducing the components. The reaction medium is stirred at 35° C. for 95 hours, then cooled to ambient temperature. Half of the mixture is treated with 150 ml of water, then acidified with 10 ml of 10% hydrochloric acid. The aqueous phase is extracted with 2×100 ml of ethyl acetate. The combined organic phases are dried over sodium sulfate Na₂SO₄, filtered and evaporated to dryness. A viscous orange liquid is obtained. TLC after acetonitrile:MeOH:CH₃COOH (96:4:1) extraction. The other half of the reaction medium will be treated in an identical manner. For purification, 32 g of viscous orange liquid are placed in a 500 ml single-necked flask and 50 ml of ethyl acetate are added. The medium is heated at 50° C. for 10 minutes and 100 ml of ethyl acetate are added to the dispersion. The mixture is heated at 50° C. for 30 minutes and the precipitate is isolated by filtration. The white solid on the filter is rinsed with small portions of ethyl acetate and will be dried at 50° C. under vacuum. The purity and structure of the white solid is confirmed by NMR analysis.

¹H RMN (600 MHz, DMSO-d6) δ 12.31 (s, 2H), 7.51-7.22 (m, 2H), 5.83 (dt, J=9.6, 2.5 Hz, 1H), 3.95 (q, J=7.0 Hz, 1H), 3.61 (ddd, J=8.3, 4.6, 2.8 Hz, 1H), 3.32 (s, 3H), 2.78 (ddd, J=7.6, 5.1, 1.9 Hz, 1H), 2.55 (m, 2H), 2.3-2.16 (m, 1H), 1.45 (ddd, J=38.7, 19.9, 4.0 Hz, 3H).

### Monomer According to the Invention, Compound (1): Example 7

### Synthesis of 3-[5-(carboxymethyl)-4-oxo-2-phenylthiazolidin-3-yl]propanoic acid by reaction of mercaptosuccinic acid, I3-alanine and benzaldehyde

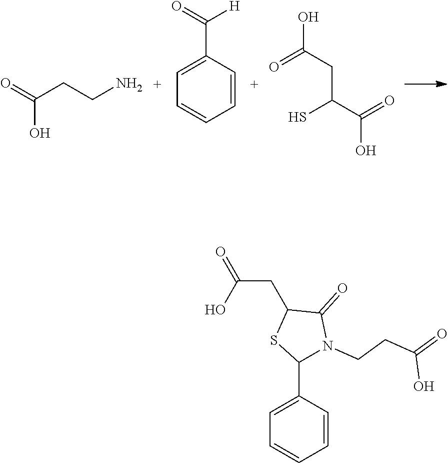

A 100 ml four-necked flask, dried beforehand under vacuum at more than 100° C. and equipped with a condenser, bubbler, thermometer and magnetic stirrer bar, is charged with 5.31 g (50 mmol) of benzaldehyde (Sigma Aldrich B1334), followed by 50 ml of demineralized water with stirring at ambient temperature. To the cloudy phase are added 4.45 g (50 mmol) of 99% β-alanine (Sigma Aldrich 05160) followed by 7.51 g (50 mmol) of thiomalic acid (Sigma-Aldrich, 88460). The reaction medium is heated at 70° C. for 71 hours before being allowed to return to ambient temperature. A 25 ml aliquot of the reaction medium is poured into a dropping funnel and then acidified with 12.5 ml of 1M hydrochloric acid. The aqueous phase is extracted with 3×20 ml of ethyl acetate. The combined organic phases are dried with sodium sulfate, filtered and evaporated. The yellow solid obtained is dissolved with the minimum amount of ethyl acetate needed and cyclohexane is added dropwise until the phase becomes very slightly cloudy. The mixture is left to stand at ambient temperature and after one night white crystals are obtained which are isolated by filtration. The crystals are washed with an ethyl acetate/cyclohexane mixture (1:1 volume) and then dried in an oven under vacuum at 50° C. The structure of the product is confirmed by proton NMR analysis and by FTIR analysis.

¹H NMR (600 MHz, DMSO-d6) δ 12.54 (s, 2H), 7.40-7.37 (m, 5H), 5.77 (d, J=1.9 Hz, 1H), 4.29 (ddd, J=8.8, 3.6, 1.9 Hz, 1H), 3.69 (dd, J=10.4, 4.7 Hz, 1H), 3.60 (ddd, J=14.5, 9.0, 5.8 Hz, 1H), 2.83-2.65 (m, 4H).

### Monomer According to the Invention, Compound (15): Example 8

### Synthesis of methyl 4-[3-[6-[2-(4-methoxycarbonylphenyl)-5-methyl-4-oxothiazolidin-3-yl]hexyl]-5-methyl-4-oxothiazolidin-2-yl]benzoate from 2-mercaptopropionic acid, hexamethylenediamine and methyl 4-formylbenzoate

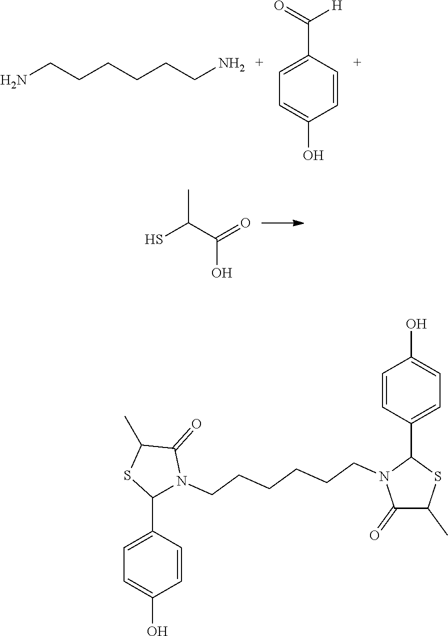

A 500 ml four-necked flask, dried beforehand under vacuum at more than 100° C. and equipped with a condenser, dropping funnel, bubbler, thermometer and magnetic stirrer bar, is charged with 28.91 g (236.75 mmol) of 4-hydroxybenzaldehyde (Acros Organics, 162772500), followed by 300 ml of ethanol with stirring at ambient temperature. Meanwhile, a solution of 13.76 g (118.37 mmol) of 98% 1,6-diaminohexane (Sigma Aldrich, H11696) in 60 ml of ethanol is prepared at 60° C. The solution is placed in the dropping funnel, and added dropwise (2 drops/second) into the reaction flask with stirring at ambient temperature. 40 ml of ethanol are used for rinsing the dropping funnel. The reaction mixture is stirred at 60° C. for 19 hours and then cooled to ambient temperature before total evaporation of the solvent. An aliquot of 11 g (33.9 mmol) of the yellow solid thus obtained is placed in a 250 ml four-necked flask dried beforehand under vacuum at more than 100° C. and equipped with a condenser, bubbler, thermometer and magnetic stirrer bar. 120 ml of N,N-dimethylacetamide are added at ambient temperature with stirring, followed by 11.36 g (101.7 mmol) of 95% 2-mercaptopropionic acid (Sigma-Aldrich, W318000). The reaction mixture is heated at 116° C. for 48 hours and then cooled to ambient temperature. The mixture is gently poured into a 2 L beaker containing 400 g of ice, with mixing with a stainless steel spatula. After an hour, a rubbery mass is deposited at the bottom of the beaker. This mass is placed in an 800 ml beaker to which 400 ml of acetone are added and stirred at ambient temperature for 30 minutes. The solid is removed by Büchner filtration and the filtrate is evaporated to dryness. The white solid obtained is dried at 50° C. under vacuum. The purity and structure of the monomer are confirmed by NMR analysis.

¹H RMN (600 MHz, DMSO-d6) δ 9.62 (s, 2H), 7.17 (s, 4H), 6.73 (s, 4H), 5.61 (s, 2H), 3.99 (d, J=78.0 Hz, 2H), 3.54-3.08 (m, 4H), 1.43 (d, J=40.7 Hz, 6H), 1.36-1.15 (m, 4H), 1.16-0.9 (m, 4H).

### Monomer According to the Invention, Compound (14): Example 9

### Synthesis of 4-[3-[6-[2-(4-carboxyphenyl)-5-methyl-4-oxothiazolidin-3-yl]hexyl]-5-methyl-4-oxothiazolidin-2-yl]benzoic acid

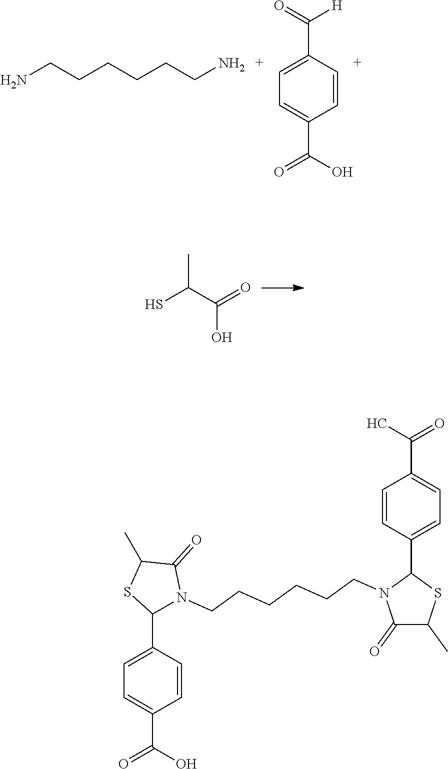

A 250 ml four-necked flask, dried beforehand under vacuum at more than 100° C. and equipped with a condenser, bubbler, thermometer and magnetic stirrer bar, is charged with 15.48 g (100 mmol) of 4-formylbenzoic acid (Sigma Aldrich 124915), followed by 60 ml of DMSO with stirring at ambient temperature. After dissolution of the 4-formylbenzoic acid, 5.81 g (50 mmol) of 1,6-diaminohexane (Sigma Aldrich, H11696) are added, followed by 40 ml of DMSO. The mixture is stirred at ambient temperature, and 11.17 g of 95% 2-mercaptopropionic acid (100 mmol) (Sigma-Aldrich, W318000) are added to the mixture rapidly and in one go. The reaction medium becomes dark orange with an increase in temperature to around 30° C. The reaction medium is stirred at ambient temperature for 70 hours; the coloration has become light orange. For purification, a 40 ml aliquot of the reaction medium is placed in a dropping funnel, followed by 150 ml of ethyl acetate and 250 ml of demineralized water. After mixing, a precipitate forms in the organic phase and the aqueous phase is removed. The organic phase and the precipitate are washed once more with 250 ml of demineralized water. After removal of the aqueous phase, the solid is isolated by filtration. The precipitate is stirred with 100 ml of ethyl acetate at ambient temperature, isolated by filtration and stirred a final time with 20 ml of ethanol. The white precipitate is isolated by filtration and dried at 80° C. in an oven under vacuum.

¹H RMN (600 MHz, DMSO-d6) δ 13.01 (s, 2H), 7.93 (dd, J=12.4, 8.3 Hz, 4H), 7.45 (dd, J=15.7, 8.3 Hz, 4H), 5.83 (d, J=6.6 Hz, 2H), 4.34-3.83 (m, 2H), 3.723.40 (m, 4H), 1.44 (dd, J=44.4, 7.0 Hz, 6H), 1.37-1.15 (m, 4H), 1.16-0.91 (m, 4H).

### Polycondensate not in Accordance With the Invention: Example 10

### Synthesis of a polycondensate by reaction of thiolactic acid (2-mercaptopropionic acid), terephthalaldehyde and hexamethylenediamine

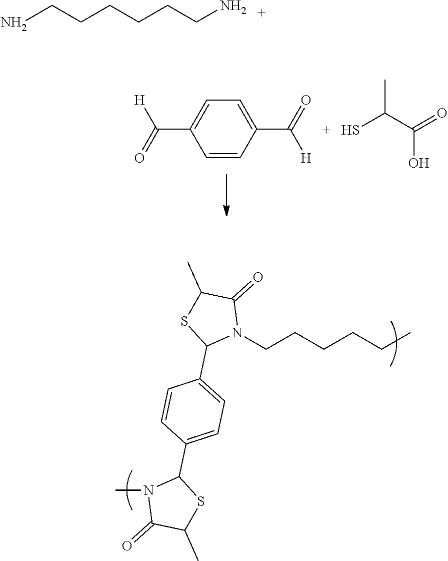

A 250 ml three-necked flask, dried and equipped with a silicone septum, is charged, with magnetic stirring and under a nitrogen atmosphere, with 1.3414 g of terephthalaldehyde (10 mmol) (Aldrich, T2207), and then 6.4 ml of dimethyl sulfoxide (DMSO). After dissolution of the aldehyde, 1.1621 g of hexamethylenediamine (10 mmol) (Merck, #8.04323.0250) dissolved in 1.6 ml of (anhydrous) DMSO are added rapidly. Immediately afterwards, 4 ml of 95% 2-mercaptopropionic acid (95%, Sigma Aldrich, W318000) are added: a white precipitate forms. After stirring overnight at 25° C. under a stream of nitrogen, the reaction medium has become transparent and light yellow without any precipitate. 500 μl of the reaction medium is taken and added to 1 ml of water: a white polymer precipitates. After removal of the milky liquid, the polymer is treated with an aqueous 1% (by mass) sodium hydroxide solution and rinsed copiously with water. The white polymer is dried under vacuum at 70° C. for 30 minutes, 150° C. for 30 minutes.

The progress of the reaction is monitored by Fourier transform infrared (FTIR): the formation of the 4-thiazolidinone unit is monitored by the appearance of a signal at 643 cm⁻¹.

The glass transition temperature determined by DSC analysis is 115° C. The polymer is thermally stable up to 350° C. The polymer is fragile and brittle: it is cracked after evaporation of the solvent.

### Polyester Polycondensate in Accordance With the Invention: Example 11

### Synthesis of a polyester polycondensate by reaction of the compound (4) monomer with itself

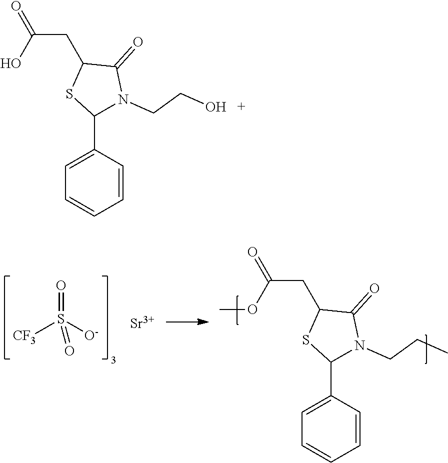

A 25 ml four-necked flask, dried beforehand under vacuum at more than 100° C. and equipped with a condenser, bubbler, thermometer and magnetic stirrer bar, is charged with 9.84 g of scandium Sc(III) triflate (i.e. 0.02 mmol), and then 563 mg of THZP-2003A dissolved in 3 ml of anhydrous acetonitrile. The mixture is stirred at 75° C. under nitrogen for 24 h. The reaction mixture is then heated to 85° C. to distil the solvent and the temperature of the reaction medium is increased to 185° C. (10° C. every 10 minutes) to complete the polymerization reaction. The polymerization is monitored by FTIR: decrease in the signal at 3600 cm⁻¹ (OH function) with the degree of polymerization of the monomer and change in absorbance of the carbonyl group from 1500 cm⁻¹ (COOH function) to 1750 cm⁻¹ (ester function).

### Polyamide Polycondensate in Accordance With the Invention: Example 12

### Synthesis of a polyamide polycondensate by reaction of the compound (3) monomer with 1,6-hexamethylenediamine

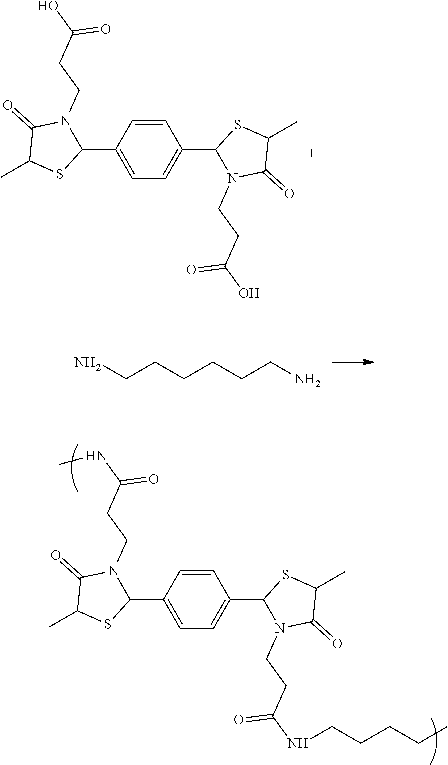

1,6-Hexamethylenediamine (HDMA, Sigma Aldrich H11696) (344.6 mg) dissolved in 1.5 ml of anhydrous ethanol is degassed under nitrogen at 60° C. Then, 134.1 mg of compound (3) dissolved in 3.5 ml of ethanol are mixed with 150 μl of HDMA at 60° C. The resulting solution is cooled in an ice bath to precipitate the salt. The crystals obtained are dried at 60° C. under vacuum and are heated in a capsule placed in a differential scanning calorimetry (DSC) apparatus under nitrogen from -80° C. to 400° C. according to a temperature ramp of 10° C./min.

DSC analysis over a temperature range extending from −80° C. to 400° C. measures, in the second cycle, a glass transition temperature of 107° C. and a first melting extending from 220° C. to around 330° C. with a peak at 288° C. and a second melting with a peak at 353° C.

### Polyurethane Polycondensate in Accordance With the Invention: Example 13

### Synthesis of a polyurethane polycondensate by reaction of the compound (5) monomer with the diisocyanate prepolymer “Adiprene LFP 950A” (PPDI prepolymer), (Chemtura, sold by Lanxess, USA)

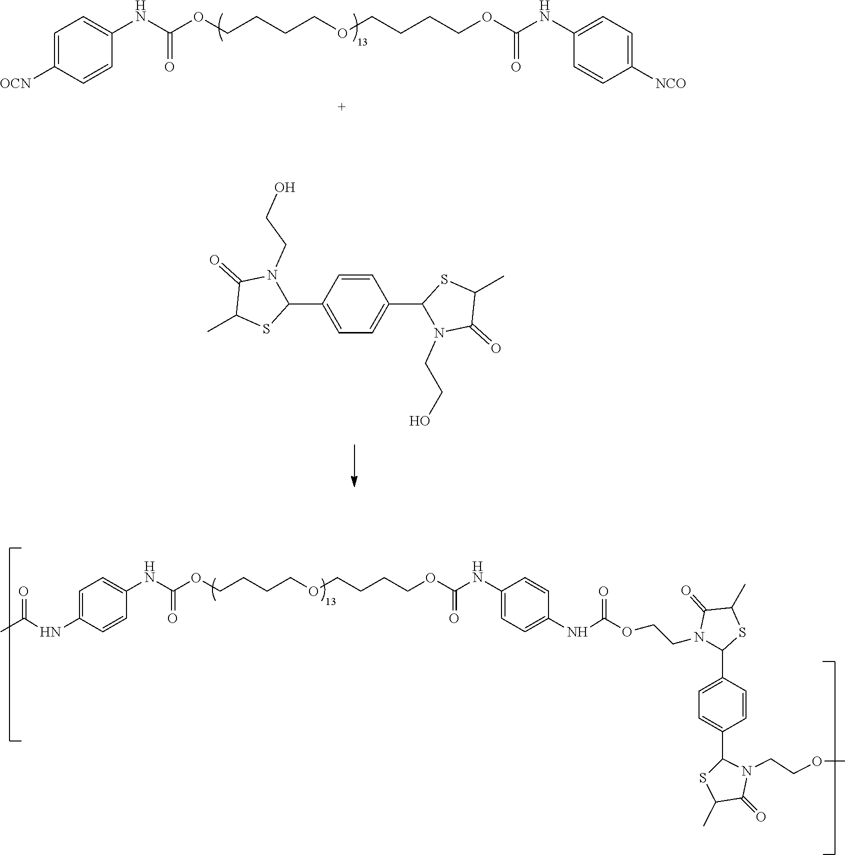

The Adiprene LFP 950A prepolymer (4.16 g) (Chemtura, Italy) is heated for 20 minutes at 80° C. Once melted, it is degassed under vacuum (10 mbar). Compound (5) (0.856 g) is added and the reaction mixture is mixed well using a spatula. The whole is placed in an oven at 110° C. and degassed once more under vacuum for 10 minutes. The product obtained is poured into a mould coated beforehand with a Gorapur L51040-33 mould release agent). The mould is covered with a (preheated) weight of 3.5 kg and placed in an oven at 150° C. for 4 hours. At the end of 4 hours, no free isocyanate remains according to FTIR analysis at 2264.5 cm⁻¹.

DSC analysis over a temperature range extending from −130° C. to 250° C. measures a glass transition temperature of −56° C. in the third cycle, and there is no melting over the temperature range swept either in the first cycle or in the second and third cycles. The absence of melting in this temperature range ensures maintenance of the moduli of the polyurethane and therefore of its mechanical properties.

DMA analysis over a temperature range extending from −120° C. to 180° C. measures a glass transition temperature (Tg) of −58° C., a Young's modulus (E′) at 25° C. of 42.7 MPa and at 50° C. of 34.1 MPa, and an elongation at break of 600%. The test specimen has the dimensions H=10, T=3.66, W=4.4.

Furthermore, no permanent set is observed for elongations of less than 10%, which means that the polyurethane is elastic at deformations of less than 10% and is therefore not subject to creep under the effect of a tensile stress.

### Polyurethane Polycondensate in Accordance With the Invention: Example 14

### Synthesis of a polyurethane polycondensate by reaction of the compound (5) monomer with the diisocyanate prepolymer “Adiprene LFM C525”, polycaprolactone α,ω-diphenylmethane diisocyanate (Chemtura, Italy) (MDI prepolymer)

The Adiprene LFM C525 prepolymer (100 parts, by mass) is heated for 20 minutes at 130° C. Once melted, it is degassed under vacuum (10 mbar). 1,4-butanediol (5.9 parts) is added and the reaction mixture is homogenized well with a mixer (“State Mix”). The whole is poured into a mould heated beforehand to 120° C. and cured for 16 hours at 120° C.

DSC analysis over a temperature range extending from −80° C. to 260° C. under nitrogen measures a glass transition temperature of −7° C. and there is no melting over the temperature range swept. The absence of melting in this temperature range ensures maintenance of the moduli of the polyurethane and therefore of its mechanical properties.

Furthermore, no permanent set is observed for elongations of less than 10%, which means that the polyurethane is elastic at deformations of less than 10% and is therefore not subject to creep under the effect of a tensile stress.

### Polyurethane Polycondensate not in Accordance With the Invention: Example 15

### Synthesis of a polyurethane polycondensate by reaction of 1,4-butanediol, a non-compliant monomer, with the diisocyanate prepolymer “Adiprene LFM C525”, polycaprolactone α,ω-diphenylmethane diisocyanate

The Adiprene LFM C525 prepolymer (100 parts, by mass) is heated for 20 minutes at 130° C. Once melted, it is degassed under vacuum (10 mbar). 1,4-butanediol (5.9 parts) is added and the reaction mixture is homogenized well with a mixer (“State Mix”). The whole is poured into a mould heated beforehand to 120° C. and cured for 16 hours at 120° C.

DSC analysis carried out over a temperature range extending from −80° C. to 250° C. under nitrogen shows that the polyurethane begins to melt starting from the ambient temperature: its moduli E′ and E″ fall, which means that the polyurethane then loses all of its mechanical properties starting from the ambient temperature.

### Polyurethane Polycondensate not in Accordance With the Invention: Example 16

### Synthesis of a polyurethane polycondensate by reaction of hydroquinone bis(2-hydroxyethyl) ether (HQEE) (Vibracure 2101), a non-compliant monomer, with the diisocyanate prepolymer “Adiprene LFM C525”

The Adiprene LFM C525 prepolymer (100 parts) (Chemtura, Italy) is heated for 20 minutes at 130° C. Once melted, it is degassed under vacuum (10 mbar). The HQEE diol (11.8 parts) is added and the reaction mixture is homogenized well with a mixer (“State Mix”). The whole is poured into a mould heated beforehand to 120° C. and cured for 16 hours at 120° C.

DSC analysis over a temperature range extending from −80° C. to 250° C. under nitrogen measures a glass transition temperature of −32° C. and melting extending from around 92° C. to around 180° C. with two melting peaks at 152° C. and at 172° C.

The first DSC cycle shows that the polyurethane is thermoplastic, since, in addition to a Tg of about −40° C., it has two other Tgs, respectively of about 45° C. and 89° C., and it begins to melt starting from around 140° C.

DMA analysis over a temperature range extending from −120° C. to 180° C. measures a glass transition temperature (Tg) of −27° C. and a Young's modulus at 50° C. of 34 MPa. The test specimen has the dimensions H=10, T=3.02, W=4.9.

Furthermore, a permanent set is observed for elongations of less than 10%, which means that the polyurethane is not elastic at deformations of less than 10% and is therefore subject to creep under the effect of a tensile stress.

### Polyurethane Polycondensate in Accordance With the Invention: Example 17

### Synthesis of a polyurethane polycondensate by reaction of the compound (5) monomer with the diisocyanate prepolymer “Adiprene LFP E560”, (Chemtura, sold by Lanxess, USA) (PPDI prepolymer)

The Adiprene LFP E560 prepolymer (100.0 g) (Chemtura, Italy) is heated for 20 minutes at 100 C. Once melted, it is degassed under vacuum (<1 mbar) until bubbles no longer form in the prepolymer (for less than 3 min). Compound (5) is added in powder form (20.76 g), dried for 16 h at 100° C., and the reaction mixture is mixed well using a mixer (“State Mix”), and then is degassed again. The product obtained is poured into a mould coated beforehand with a “BYK A-501” mould release agent sold by the company BYK-Chemie, Germany. The mould is covered with a (preheated) weight of 3.5 kg and placed in an oven at 150° C. for 4 hours. At the end of 4 hours, no free isocyanate remains according to FTIR analysis at 2264.5 cm⁻¹.

DSC analysis over a temperature range extending from −130° C. to 250° C. measures a glass transition temperature of −48.8° C. in the second cycle and there is no melting over the temperature range swept in the second cycle. The absence of melting in this temperature range ensures maintenance of the moduli of the polyurethane and therefore of its mechanical properties.

DMA analysis over a temperature range extending from −120° C. to 180° C. measures a glass transition temperature (Tg) of −40° C., a Young's modulus (E′) at 25° C. of 75.7 MPa and of 59.2 MPa at 100° C.

The results show that the monomers according to the invention provide access to a wide variety of polyamide, polyester and polyurethane polycondensates, and do so without the use during the polycondensation reaction of an odorous thiol, such as 2-mercaptopropionic acid.

The results also show that the polycondensates according to the invention are much less fragile than the polycondensate obtained by polycondensation of 2-mercaptopropionic acid, hexamethylenediamine and terephthalaldehyde and described in the journal Polymer Chemistry, 2014, 5, 2695.

The results show that the polyurethanes containing 4-thiazolidinone units in their monomer units exhibit properties superior to those of the polyurethanes without 4-thiazolidinone units within their chain.

Specifically, the absence of melting of the polyurethanes according to the invention at temperatures as high as 250° C. ensures that their mechanical properties are maintained at these temperatures, which is not the case for the polyurethanes without a 4-thiazolidinone unit within their chain.

Furthermore, the polyurethanes according to the invention have the advantage over their non-compliant counterparts of having an elastic behaviour at deformations of less than 10%, which also ensures dimensional stability of any part made from polyurethane according to the invention and subjected to deformations of less than 10%.

The improvement in the properties is attributed to the presence of the 4-thiazolidinone units within the polymer chain of the polyamide, polyester and polyurethane polycondensates.

1. A monomer, comprising:
a 4-thiazolidinone unit connected to a benzene nucleus and two functions selected from hydroxyl and carboxyl, which monomer is of formula (I-a) or (I-b) in which Y is hydroxyl or carboxyl, Q is alkyl or carboxyalkyl, R₂ is hydrogen, hydroxyl, carboxyl, alkyl or hydroxyalkyl, Y′ is hydroxyl or carboxyl; Q′ is alkyl or carboxyalkyl, 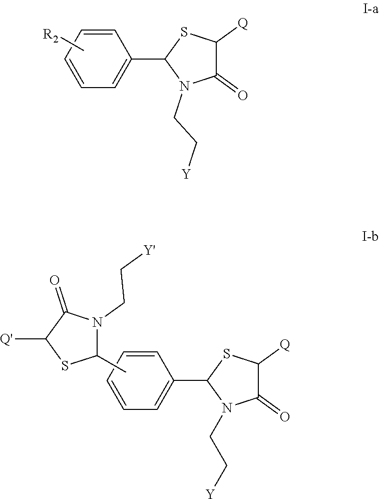
or of formula (I-c) in which Y is hydroxyl or carboxyl, Q is alkyl or carboxyalkyl, Y′ is hydroxyl or carboxyl; Q′ is alkyl or carboxyalkyl, x is an integer ranging from 0 to 2, 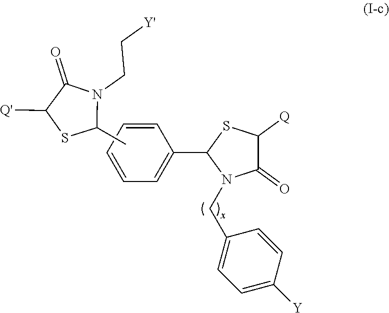
or else of formula (II-1) in which Q is alkyl or carboxyalkyl, R₃ is hydrogen, hydroxyl, carboxyl, alkyl or hydroxyalkyl, R₁ is alkyl and A₁, a divalent carbon group which contains carbon atoms, is an alkanediyl or an alkanediyl interrupted by one or more oxygen atoms, m is an integer ranging from 0 to 4

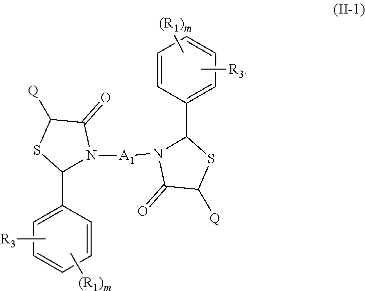

2. The monomer according to claim 1, wherein m is equal to 0.

3. The monomer according to claim 1, wherein the alkyls represented by Q and Q′ are methyl, the carboxyalkyls represented by Q and Q′ are carboxymethyl, the hydroxyalkyls represented by R₂ and R₃ are hydroxymethyl.

4. The monomer according to claim 1, wherein R₂ is hydrogen, hydroxyl, carboxyl or hydroxymethyl.

5. A process for synthesizing a monomer defined in claim 1 which comprises the reaction of an aldehyde of formula (II), a compound (III) and a compound of formula (IV) 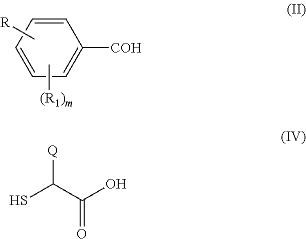
the compound (III) being of formula Y-A-NH₂ or of formula H₂N-A₁-NH₂,
in which Y is hydroxyl or carboxyl, Q is alkyl or carboxyalkyl, A is an ethanediyl, A₁ is an alkanediyl or an alkanediyl interrupted by one or more oxygen atoms, R₁ is alkyl, m is an integer ranging from 0 to 4, R is hydrogen, hydroxyl, carboxyl, alkyl, hydroxyalkyl or an aldehyde function.

6. A process for synthesizing a polyamide which comprises the reaction of a monomer defined in claim 1 with a second monomer containing two functions selected from amine and isocyanate, the monomer containing two carboxyl functions.

7. A process for synthesizing a polyester which comprises the reaction of a monomer defined in claim 1 with a second monomer, both monomers each containing a carboxyl function and a hydroxyl function or alternatively one of the two monomers containing two hydroxyl functions and the other containing two carboxyl functions.

8. A process for synthesizing a polyurethane which comprises the reaction of a monomer defined in claim 1 with a second monomer containing two isocyanate functions, the monomer containing two hydroxyl functions.

9. A process according to claim 6, wherein the second monomer is partly or totally replaced by a prepolymer which contains two reactive functions identical to that of the second monomer.

10. A polycondensate capable of being obtained by a process defined in claim 6, the polycondensate being a polyamide, a polyester or a polyurethane and containing monomer units comprising 4-thiazolidinone units.
